SEQUENCE LISTING (8 sequences)

SEQ ID NO: 1 - RNA, 21 nt, Artificial
  synthetic nucleic acid
cuagccauggcguuaguauuu

SEQ ID NO: 2 - RNA, 19 nt, Artificial
  synthetic nucleic acid
ucaacugacucgaccacua

SEQ ID NO: 3 - RNA, 21 nt, Artificial
  synthetic nucleic acid
ggaaggugcuuguggauauuu

SEQ ID NO: 4 - RNA, 19 nt, Artificial
  synthetic nucleic acid
gggccuaggaccuguagua

SEQ ID NO: 5 - RNA, 19 nt, Artificial
  synthetic nucleic acid
gcgaaggcguccacaguua

SEQ ID NO: 6 - RNA, 21 nt, Artificial
  synthetic nucleic acid sequences
agucacggcuagcugugaauu

SEQ ID NO: 7 - DNA, 18 bp, Artificial
  synthetic nucleic acid
ggaaggtcctgtggacat

SEQ ID NO: 8 - DNA, 19 bp, Artificial
  synthetic nucleic acid
atatccacaagcaccttcc

(19) United States
(12) Patent Application Publication
(10) Pub. No.: US 2011/0305772 A1
(43) Pub. Date: Dec. 15, 2011

(54) COMPOSITIONS AND METHODS FOR EX VIVO HEPATIC NUCLEIC ACID DELIVERY

(75) Inventor: Andrew M. Cameron, Baltimore, MD (US)

(73) Assignee: THE JOHNS HOPKINS UNIVERSITY, Baltimore, MD (US)

(21) Appl. No.: 13/203,618

(22) PCT Filed: Feb. 26, 2010
(86) PCT No.: PCT/US10/25505

§ 371 Date: Aug. 26, 2011

Related U.S. Application Data
(60) Provisional application No. 61/155,661, filed on Feb. 26, 2009.

(51) Int. Cl. A61K 35/407; A01N 1/02; A61P 1/16; C12N 5/07

(52) U.S. Cl. 424/553; 435/375; 435/1.2

(57) ABSTRACT

The invention provides compositions and methods for delivery of nucleic acids to the liver ex vivo. The method includes slow, low pressure infusion of the nucleic acid into the liver to efficiently transduce cells with minimal cell damage.

### REFERENCE TO RELATED APPLICATIONS

This application claims priority to U.S. Provisional Patent Application Ser. No. 61/155,661; filed Feb. 26, 2009, which is incorporated herein by reference in its entirety.

### BACKGROUND

Hydrodynamic and virally mediated nucleic acid delivery are potentially useful techniques for therapeutic nucleic acid applications. Hydrodynamic nucleic acid delivery was first used to demonstrate that siRNA-mediated gene expression knockdown was feasible in adult mammals. Virally mediated nucleic acid delivery has long been known and many gene therapy trials using viral delivery of nucleic acids are underway.

Several reports have appeared using the techniques to deliver siRNA to various organs, especially rodent livers. siRNA holds great promise as a therapeutic tool but delivery has remained an unsolved hurdle in clinical scenarios. Potential applications of siRNA directed against various targets in diseases, for example cancer, infectious disease, or dominant genetic disease, are conceivable.

### SUMMARY OF THE INVENTION

The invention provides methods for delivering nucleic acids to the liver ex vivo prior to transplant. The invention further provides compositions for delivery to the liver ex vivo prior to transplant.

The invention provides methods of ex vivo hepatic nucleic acid delivery including the steps of obtaining a liver; and injecting the nucleic acid in a carrier into vasculature of the liver at a low pressure about 2 mm Hg to about 15 mmHg, whereby the nucleic acid is delivered to hepatic tissue. Low pressure includes a pressure of about 2, 3, 4, 5, 6, 7, 8, 9, 10, 11, 12, 13, 14, or 15 mmHg, or any range bracketed by those values. In an embodiment, low pressure is about 2 to 10 mm Hg. In an embodiment, low pressure is about 5 to 12 mm Hg. In an embodiment, low pressure is about 8 to 12 mm Hg. In an embodiment, low pressure is about 5 to 11 mm Hg.

In the methods provided, the volume of the nucleic acid in the carrier to be delivered is small as compared to the volume of the liver, and will be determined based on the weight of the liver. In an embodiment, about 0.5 ml to about 30 ml of nucleic acid is provided to an adult liver which is typically about 1.5 kg (that is about 0.33 ml/to about 20 ml/kg of liver to be transplanted). The volume to be delivered to a pediatric liver can be determined proportionately. That is the volume to be delivered is about 0.5, 1, 2, 3, 4, 5, 6, 7, 8, 9, 10, 11, 12, 13, 14, 15, 16, 17, 18, 19, or 20 ml, or any range bracketed by those values, per kg of recipient liver. In certain embodiments, the volume delivered is about 1 ml to about 20 ml. In certain embodiments, the volume delivered is about 0.5 ml to about 10 ml. In certain embodiments, the volume delivered is about 5 ml to about 20 ml. In certain embodiments, the volume delivered is about 1 ml to about 15 ml. In certain embodiments, the volume delivered is about 5 ml to about 20 ml. In certain embodiments, outflow of fluid from the liver can be prevented, for example, by clamping the appropriate arteries and/or veins depending on the direction of injection. In certain embodiments, the nucleic acid and carrier can be perfused through or recirculated through the liver. In such embodiments, the total volume of nucleic acid in carrier passing through the liver is greater than 30 ml, however, the total volume of perfusate in the liver at any one time is preferably no greater than about 30 ml for a 1.5. kg liver.

To maintain the low delivery pressure, the volume of the nucleic acid in carrier will be delivered relatively slowly. For example, in methods of the invention, the nucleic acid in the carrier is injected at a rate of about 2 to about 15 ml/minute, that is about 2, 3, 4, 5, 6, 7, 8, 9, 10, 11, 12, 13, 14, or 15 ml/minute, or any range bracketed by any of those values. In certain embodiments, the delivery rate is about 2 ml to about 10 ml/minute. In certain embodiments, the delivery rate is about 5 ml to about 10 ml/minute. In certain embodiments, the delivery rate is about 5 ml to about 15 ml/minute.

The methods of the invention provide for perfusing the liver with a pharmaceutically acceptable carrier, preferably a solution used in conjunction with liver transplantation, prior to delivery of the nucleic acid in carrier. In certain embodiments, the method provides perfusion of the liver with a pharmaceutically acceptable carrier prior to the injection of the nucleic acid. In certain embodiments, the method provides for perfusion of the liver with a cold pharmaceutically acceptable carrier prior to injection of the nucleic acid in the carrier.

Methods of the invention allow for perfusion of the liver with a the portal vein, hepatic artery, or inferior vena cava. In certain embodiments, the nucleic acid in the carrier is perfused through the liver in the same direction (i.e., in relation of direction of flow through the vasculature, forward or retrograde) as the carrier without nucleic acid was perfused. In certain embodiments, the nucleic acid in the carrier is perfused through the liver in the opposite direction as the carrier without nucleic acid was perfused.

In certain embodiments, the nucleic acid in the carrier is retained in the liver for a period of time prior to transplantation of the liver and perfusion of the liver with blood, or a pharmaceutically acceptable carrier without nucleic acid. The amount of time that the nucleic acid is retained in the liver is largely determined by the liver transplant procedure. In certain embodiments, the liver is contacted with the nucleic acid ex vivo in the pharmaceutical carrier for at least 10 minutes. In certain embodiments, the liver is contacted with the nucleic acid ex vivo in the pharmaceutical carrier for at least 15 minutes. In certain embodiments, the liver is contacted with the nucleic acid in the pharmaceutical carrier ex vivo for at least 30 minutes. In certain embodiments, the liver is contacted with the nucleic acid in the pharmaceutical carrier ex vivo for at least 45 minutes. In certain embodiments, the liver is contacted with the nucleic acid in the pharmaceutical carrier ex vivo for at least 60 minutes. The maximum amount of time that the nucleic acid and carrier can be contacted with the liver ex vivo is controlled substantially by the liver transplant process. It is advantageous to transplant the liver into the recipient as quickly as is reasonably possible, and preferably no longer than 12 hours from when the liver is removed from the donor. Therefore, the liver would be contacted with the nucleic acid in the carrier for no more than 12 hours.

The methods of the invention provide for the delivery of essentially any type of nucleic acid. In certain embodiments, the nucleic acid is an siRNA. In certain embodiments, the nucleic acid is an antisense nucleic acid. In certain embodiments, the nucleic acid is an shRNA.

The nucleic acid can be designed for essentially any therapeutic purpose. The invention includes the nucleic acid sequences and constructs provided herein for use in the context of the invention. For example, the nucleic acid can be targeted to a hepatitis virus, such as a hepatitis C virus. In certain embodiments, the nucleic acid specifically hybridizes to at least 15 contiguous nucleic acids of a sequence of one of:

| | |
|---|---|
| 5′-CUAGCCAUGGCGUUAGUAUUU-3′, | (SEQ ID NO: 1) |
| 5′-UCAACUGACUCGACCACUA-3′, | (SEQ ID NO: 2) |
| 5′-GGAAGGUGCUUGUGGAUAUUU-3′, | (SEQ ID NO: 3) |
| 5′-GGGCCUAGGACCUGUAGUA-3′, | (SEQ ID NO: 4) |
| 5′-GCGAAGGCGUCCACAGUUA-3′, | (SEQ ID NO: 5) |
| and | |
| 5′-AGUCACGGCUAGCUGUGAAUU-3′. | (SEQ ID NO: 6) |

In certain embodiments of the invention, the antisense nucleic acid, siRNA or the shRNA is encoded by an expression construct. In certain embodiments, the expression construct is present in a vector. Vectors include, but are not limited to plasmid vectors, an adenoviral (Ad) vectors, an adeno-associated viral vectors (AAV), a lentiviral vectors, and a herpes simplex viral (HSV) vectors. In certain embodiments, the AAV viral vector is a serotype for infection of liver cells such as an AAV2 viral vector, an AAV8 vector, an AAV9 vector, a hybrid AAV2/4 viral vector, and a hybrid AAV2/5 viral vectors. In certain embodiments, the AAV viral vector is self-complementary. In certain embodiments, the viral vector is replication competent. In certain embodiments, the viral vector is replication incompetent.

The invention provides for the use of any promoter or other transcriptional regulatory sequences that are active in liver cells. For example, expression constructs can include a promoter sequence such as a cytomegalovirus (CMV) promoter, a β-globin promoter, chicken β-actin (CBA) promoter, and small chicken β-actin (smCBA) promoter, and an EF-1α promoter.

The invention provides for the delivery of any amount of nucleic acid that provides the desired therapeutic effect. In certain embodiments, the invention provides for the deliver of about 1×10¹⁰ to 1×10¹⁴ viral particles per 1.5 kg liver, that is 0.67×10¹⁰ to 0.67×10¹⁴ viral particles per kg of liver. When non-viral nucleic acid is to be delivered to the liver, the invention provides for doses of about 50 mg to 1000 mg nucleic acid (e.g., plasmid, siRNA, shRNA, antisense nucleic acid) per 1.5 kg liver, that is about 33 mg to about 667 mg of nucleic acid per kg of liver.

In certain embodiments of the invention, the liver is obtained from a cadaveric donor. In certain embodiments of the invention, the liver is obtained from a living donor (e.g., single lobe transplant in pediatric transplants).

The methods of the invention further provide for transplanting the liver into a subject after transfer of the nucleic acid.

In certain embodiments, the invention further includes monitoring the subject for HCV infection.

In certain embodiments, the invention provides for administration of an inhibitor of intracellular adhesion molecule-1 to the liver, for example, a nucleic acid targeted to intracellular adhesion molecule-1. In certain embodiments, the invention provides for administration of an inhibitor of mir-122 to the liver, such as a nucleic acid targeted to mir-122.

The invention further provides kits for practicing the methods of the invention.

Other embodiments of the invention are provided infra.

### BRIEF DESCRIPTION OF THE FIGURES

FIG. 1. Picture of PV-slow injection. 18 G catheter is inserted in the portal vein. After the volume was passed slowly, the suprahepatic (*) and infrahepatic IVC (**) were clamped 50% of the liver weight volume is injected slowly maintaining the pressure between 10 to 13 mm Hg. A manometer (06-664-18, Fisher Scientific inc., Newark, Nebr., USA) was utilized with a three-way stopcock. After injection, clamps were promptly removed.

FIG. 2. Delivery of Cy3 labeled ICAM siRNA (red) into cold preserved liver graft by various hydrodynamic injections. (A) Tissue sections were collected at 1 hour after hydrodynamic injection and counterstained with Alexa488-phalloidin (green). The merged images of the two channels are shown. Two rapid injection groups showed strong and intense uptake in cytoplasm and nucleus (bright dot in the center of cell). However, PV slow injection group showed relatively diffuse and weak staining. Many periportal vein non-parenchymal cells (arrows) and perihepatic vein or sinusoidal endothelial cells (arrow heads) were positive in all groups. (B) Delivery area (%) was similar between the three different injection methods.

FIG. 3. Trypan blue uptake after various hydrodynamic injections. (A) 40-50 ml of 200 mmol cold Trypan blue solution was perfused via the portal vein of the liver graft at 1 hour after hydrodynamic injection, followed by 30-40 ml of cold 4% paraformaldehyde perfusion 5 mm frozen sections were cut and counterstained with Eosin. Three representative fields were selected from each lobe and Trypan blue positive cells were counted under the microscope (×200). The mean number of positive cells from three different lobes was considered as overall positive number per high power field (HPF). Many Trypan blue uptake cells were observed in rapid injection groups. (B) Most Trypan blue positive cells were sinusoidal endothelial cells (arrows). There was no difference between control (no injection) and the slow injection group. However, the rapid injection groups showed more Trypan blue positive cells than control and PV slow injection group.

FIG. 4. Degree of injury at 24 hours after transplantation. (A) Representative H&E staining showed several focal necroses around portal veins in the rapid injection group 24 hours after transplantation. In the slow injection group, very few small focal necroses were observed. (B) In TUNEL staining, several TUNEL positive cells were observed in hepatocytes and endothelial cells around the portal vein. (C) However, few TUNEL positive cells were observed in the slow group. (D) Mean ALT levels 24 hours after transplantation were less than 1,000 U/dl in both groups, however, the slow injection group showed lower ALT levels than the rapid group.

FIG. 5. Functional outcomes at 24 hours after transplantation according to injection methods. Hepatocytes were isolated from transplanted livers using a two-step collagenase perfusion and differential centrifugation technique. Cyclophylin B mRNA relative expression levels were assayed by RT-PCR using RNA from hepatocytes. LacZ siRNA treated animals using the same delivery method (6 animals each) were used as control for each group. PV-slow group showed better functional knockdown than the PV rapid group. The PV-slow group showed 69.8±51.7% knockdown of Cyclophylin B mRNA, as compared to 23.7±46.0% in the PV-rapid group.

FIGS. 6A-C. HCV target knockdown and prevention of infection using siRNA. (A) shows siRNA sequences and the locus to which they are targeted; (B) shows the three day results of quantitative PCR based assays of cellular HCV levels in response to treatment with our anti-HCV siRNAs indicated. Several were potent in inhibiting HCV replication on the order of 95-99%; (C) shows the three day results of quantitative PCR based assays of cellular HCV demonstrating protection of cells by siRNA prior to infection with HCV.

FIGS. 7A-B. Generation of antiHCV shRNAs. (A) shows a schematic diagram of Hepatitis C viral genome with an arrow indicating the location of the NS4 target sequence. (B) shows a schematic of the AAV viral vector construct design. The shRNA show the coding sequences are inserted in the GFP intronic sequence. NS4 shRNA sense sequence is 5′-GGAAGGTCCTGTGGACAT-3′ (SEQ ID NO: 7) (underlined), and the antisense sequence is 5′ATATCCACAAGCACCTTCC-3′ (SEQ ID NO: 8) (italics). The intervening repeated N sequence can be any sequence that does not interfere with hybridixation of the sense sequence with the antisense sequence.

FIG. 8A-C. Expression of siRNAs of interest. (A) shows microRNA expression in clone B cells after transient transfection of pAAVeGFP-HBV and pAAVeGFP-NS4 plasmids. RNA was isolated at 1, 2, and 3 days post transfection. MicroRNA northern blots were hybridized with (A) HBV and (B) NS4 oligonucleotide probes as indicated. (C) Blot probed with miR16 as a loading control.

FIG. 9A-D. Transfection of human hepatocytes with anti-HCV siRNA. Efficiency of transfection of clone B cells with pAAVeGFP plasmids. The figure shows (A) brightfield, (B) green channel immunofluorescence, and (C) a merged brightfield and GFP image. (D) Clone B replicon analysis was performed on total RNA isolated from clone B cells and parental cells (Huh-7). Northern blot was performed using a replicon-specific probel for the NS5b sequence is shown. Blot was stripped and reprobed for GAPDH transcript as a loading control.

FIG. 10A-B. Decreased HCV levels in response to anti-HCV shRNA. Knockdown of HCV replicon transcript expression in clone B cells transiently transfected with pAAVeGFP-NS4 plasmid. (A) Total RNA was isolated 1, 2, and 3 days after transfection. Northern blots were hybridized with NS5b probe (upper panel). The lower panel shows 18S RNA in an ethidium bromide stained gel prior to transfer. (B) Quantitative (q)PCR was performed to detect the HCV transcript. Total RNA was isolated from clone B cells three days after transient transfection with pAAVeGFP-HBV and pAAVeGFP-NS4 plasmid. RT-qPCR was performed. HCV replicon transcript expression in NS4-transfected cells is normalized to HBV-transfected cells.

FIG. 11. Expression of eGFP in mouse liver after AAV-eGFP delivery. Mice were injected via tail vein with 10¹² AAV viral particles containing an scAAV8-EF1α-eGFP expression construct. After 8 weeks, livers were observed for eGFP expression. The top panels show livers of a control animal not injected with the AAV epression construct. The bottom panels show (from left to right) a color image of the liver, a fluorescent image of the liver showing GFP expression, a black and white image of the liver, and a merged image of the GFP expression and the black and white liver image.

### DETAILED DESCRIPTION

### Definitions

An “agent” is understood herein to include a therapeutically active compound or a potentially therapeutic active compound, e.g., an immunosuppressant drug. An agent can be a previously known or unknown compound. As used herein, an agent is typically a non-cell based compound, however, in certain embodiments, an agent can include a biological therapeutic agent, e.g., peptide or nucleic acid therapeutic, e.g., siRNA, shRNA, cytokine, antibody, etc.

As used herein “amelioration” or “treatment” is understood as meaning to lessen or decrease at least one sign, symptom, indication, or effect of a specific disease or condition. For example, amelioration or treatment of hepatitis, such as hepatitis C can be to reduce, delay, or eliminate one or more signs or symptoms of hepatitis C. Signs or symptoms of hepatitis C include, but are not limited to, decreased hepatic function, jaundice, and scarring of the liver. Amelioration and treatment can require the administration of more than one dose of an agent, either alone or in conjunction with other therapeutic agents and interventions. Amelioration or treatment does not require that the disease or condition be cured or prevented from recurring during the lifetime of the subject.

As used herein, “changed as compared to a control” sample or subject is understood as having a level of the analyte or diagnostic or therapeutic indicator to be detected at a level that is statistically different than a sample from a normal, untreated, or control sample. Control samples include, for example, cells in culture, one or more laboratory test animals, or one or more human subjects. Methods to select and test control samples are within the ability of those in the art. An analyte can be a naturally occurring substance that is characteristically expressed or produced by the cell or organism (e.g., viral particles, indicators of hepatic function, e.g., ALT, AST) or a substance produced by a reporter construct (e.g, β-galactosidase or luciferase). The amount of damage can be changed relative to a control, for example by biopsy and determining cell viability (e.g., trypan blue staining, apoptosis by TUNEL assay) and/or by determining liver enzyme levels. Depending on the method used for detection the amount and measurement of the change can vary. Changed as compared to a control reference sample can also include a change in liver function, virus production, etc. Determination of statistical significance is within the ability of those skilled in the art.

“Co-administration” as used herein is understood as administration of one or more agents to a subject such that the agents are present and active in the subject at the same time. Co-administration does not require a preparation of an admixture of the agents or simultaneous administration of the agents.

“Contacting a cell” is understood herein as providing an agent to a test cell e.g., a cell to be treated in culture, ex vivo, or in an animal, such that the agent can interact with the test cell or cell to be treated, potentially be taken up by the test cell or cell to be treated, and have an effect on the test cell or cell to be treated. The agent or isolated cell can be delivered to the cell directly (e.g., by addition of the agent to culture medium or by injection into the cell or tissue of interest), or by delivery to the organism by an enteral or parenteral route of administration for delivery to the cell by vascular, lymphatic, or other means. A cell can be contacted ex vivo by delivery of the agent to the tissue, for example, through the vasculature present in the tissue, by topical application, by delivery to any lumen of the tissue. Contacting can include circulation of the agent in a carrier through the tissue.

As used herein, “detecting”, “detection” and the like are understood that an assay performed to determine one or more characteristics of a sample. For example, detection can include identification of a specific analyte in a sample, a product from a reporter construct or heterologous expression construct (e.g., viral vector) in a sample, or an activity of an agent in a sample. Detection can include the determination of nucleic acid or protein expression or dye uptake in a cell or tissue, e.g., as determined by PCR, immunoassay, microscopy. Detection can include determination of cell viability/apoptosis, liver enzyme (e.g., ALT, AST, alkaline phosphatase, bilirubin) level, bile leakage, portal vein stricture, hepatic artery thrombosis, etc. The amount of analyte or activity detected in the sample can be none or below the level of detection of the assay or method.

By “diagnosing” as used herein refers to a clinical or other assessment of the condition of a subject based on observation, testing, or circumstances for identifying a subject having a disease, disorder, or condition based on the presence of at least one sign or symptom of the disease, disorder, or condition. Typically, diagnosing using the method of the invention includes the observation of the subject for other signs or symptoms of the disease, disorder, or condition.

The terms “effective amount,” or “effective dose” refers to that amount of an agent to produce the intended pharmacological, therapeutic or preventive result. The pharmacologically effective amount results in the amelioration of one or more signs or symptoms of a disease or condition or the advancement of a disease or condition, or causes the regression of the disease or condition. For example, a therapeutically effective amount preferably refers to the amount of a therapeutic agent that decreases the recurrence of the disease or condition in the subject. In the case of hepatitis, for example hepatitis C, an effective dose delays the infection of the liver with hepatitis C, particularly in the absence of de novo infection (e.g., from an external source) with hepatitis C after transplantation, for at least 0.5 years, 1 year, 1.5 years, 2 years, 3 years, 4 years, 5 years, at least 6 years, at least 7 years, at least 8 years, at least 9 years, or at least 10 years. More than one dose may be required to provide an effective dose.

As used herein, the terms “effective” and “effectiveness” includes both pharmacological effectiveness and physiological safety. Pharmacological effectiveness refers to the ability of the treatment to result in a desired biological effect in the patient. Physiological safety refers to the level of toxicity, or other adverse physiological effects at the cellular, organ and/or organism level (often referred to as side-effects) resulting from administration of the treatment. On the other hand, the term “ineffective” indicates that a treatment does not provide sufficient pharmacological effect to be therapeutically useful, even in the absence of deleterious effects, at least in the unstratified population. (Such a treatment may be ineffective in a subgroup that can be identified by the expression profile or profiles.) “Less effective” means that the treatment results in a therapeutically significant lower level of pharmacological effectiveness and/or a therapeutically greater level of adverse physiological effects, e.g., greater liver toxicity.

Thus, in connection with the administration of a drug, a drug which is “effective against” a disease or condition indicates that administration in a clinically appropriate manner results in a beneficial effect for at least a statistically significant fraction of patients, such as a improvement of symptoms, a cure, a reduction in disease signs or symptoms, extension of life, improvement in quality of life, or other effect generally recognized as positive by medical doctors familiar with treating the particular type of disease or condition.

“Expression construct” as used herein is understood as a nucleic acid sequence including a sequence for expression as a polypeptide or nucleic acid (e.g., siRNA, shRNA) operably linked to a promoter and other essential regulatory sequences to allow for the expression of the polypeptide in at least one cell type. In a preferred embodiment, the promoter and other regulatory sequences are selected based on the cell type in which the expression construct is to be used. Selection of promoter and other regulatory sequences for protein expression are well known to those of skill in the art. An expression construction preferably also includes sequences to allow for the replication of the expression construct, e.g., plasmid sequences, viral sequences, etc. For example, expression constructs can be incorporated into replication competent or replication deficient viral vectors including, but not limited to, adenoviral (Ad) vectors, adeno-associated viral (AAV) vectors of all serotypes, self-complementary AAV vectors, and self-complementary AAV vectors with hybrid serotypes, self-complementary AAV vectors with hybrid serotypes and altered amino acid sequences in the capsid that provide enhanced transduction efficiency, lentiviral vectors, or plasmids for bacterial expression.

As used herein, “heterologous” as in “heterologous protein” is understood as a nucleic acid or protein not natively expressed in the cell in which it is expressed, or a nucleic acid or protein expressed from a nucleic acid that is not endogenous to the cell. For example, a heterologous protein is a protein expressed from a reporter construct, or a protein or nucleic acid present in the cell that is expressed from an expression construct introduced into the cell, e.g. viral vector expression construct.

As used herein, the terms “identity” or “percent identity”, refers to the subunit sequence similarity between two polymeric molecules, e.g., two polynucleotides or two polypeptides. When a subunit position in both of the two molecules is occupied by the same monomeric subunit, e.g., if a position in each of two peptides is occupied by serine, then they are identical at that position. The identity between two sequences is a direct function of the number of matching or identical positions, e.g., if half (e.g., 5 positions in a polymer 10 subunits in length), of the positions in two peptide or compound sequences are identical, then the two sequences are 50% identical; if 90% of the positions, e.g., 9 of 10 are matched, the two sequences share 90% sequence identity. The identity between two sequences is a direct function of the number of matching or identical positions. Thus, if a portion of the reference sequence is deleted in a particular peptide, that deleted section is not counted for purposes of calculating sequence identity. Identity is often measured using sequence analysis software e.g., BLASTN or BLASTP (available at (www.ncbi.nih.gov/BLAST). The default parameters for comparing two sequences (e.g., “Blast”-ing two sequences against each other), by BLASTN (for nucleotide sequences) are reward for match=1, penalty for mismatch=−2, open gap=5, extension gap=2. When using BLASTP for protein sequences, the default parameters are reward for match=0, penalty for mismatch=0, open gap=11, and extension gap=1. Additional, computer programs for determining identity are known in the art.

As used herein, “isolated” or “purified” when used in reference to a polypeptide means that a naturally polypeptide or protein has been removed from its normal physiological environment (e.g., protein isolated from plasma or tissue) or is synthesized in a non-natural environment (e.g., artificially synthesized in an in vitro translation system or using chemical synthesis). Thus, an “isolated” or “purified” polypeptide can be in a cell-free solution or placed in a different cellular environment (e.g., expressed in a heterologous cell type). The term “purified” does not imply that the polypeptide is the only polypeptide present, but that it is essentially free (about 90-95%, up to 99-100% pure) of cellular or organismal material naturally associated with it, and thus is distinguished from naturally occurring polypeptide. Similarly, an isolated nucleic acid is removed from its normal physiological environment. “Isolated” when used in reference to a cell means the cell is in culture (i.e., not in an animal), either cell culture or organ culture, of a primary cell or cell line. Cells can be isolated from a normal animal, a transgenic animal, an animal having spontaneously occurring genetic changes, and/or an animal having a genetic and/or induced disease or condition. An isolated virus or viral vector is a virus that is removed from the cells, typically in culture, in which the virus was produced.

As used herein, “kits” are understood to contain at least one non-standard laboratory reagent for use in the methods of the invention. For example, a kit can include an siRNA or an expression construct for expression of an shRNA in appropriate packaging. The kit can further include any other components required to practice the method of the invention, as dry powders, concentrated solutions, or ready to use solutions. In some embodiments, the kit comprises one or more containers that contain reagents for use in the methods of the invention; such containers can be boxes, ampules, bottles, vials, tubes, bags, pouches, blister-packs, or other suitable container forms known in the art. Such containers can be made of plastic, glass, laminated paper, metal foil, or other materials suitable for holding reagents.

As used herein, “nucleic acid” as in a nucleic acid for delivery to a cell ex vivo is understood by its usual meaning in the art as a polynucleotide or oligonucleotide which refers to a string of at least two base-sugar-phosphate combinations. Nucleotides are the monomeric units of nucleic acid polymers. The term includes deoxyribonucleic acid (DNA) and ribonucleic acid (RNA) in the form of an oligonucleotide messenger RNA, anti-sense, plasmid DNA, parts of a plasmid DNA or genetic material derived from a virus. An oligonucleotide is distinguished, here, from a polynucleotide by containing less than 120 monomeric units. Polynucleotides include nucleic acids of at least two monomers. Anti-sense is a polynucleotide that interferes with the function of DNA, or more typically RNA. An siRNA or an shRNA is a double stranded RNA that inhibits or disrupts activity or translation, for example by promoting degradation of modifying splicing or processing of the cellular nucleic acid, e.g., mRNA, microRNA, to which it is targeted. As used herein, siRNA and shRNA include any double stranded RNA molecule wherein about 18 to about 30 nucleotides form the double stranded portion of the molecule wherein the double stranded RNA can modulate the stability, translation, or splicing of an RNA to which at least one strand of the double stranded nucleic acid hybridizes. RNAs are well known in the art, e.g., see patent publications WO02/44321, WO/2003/099298, US 20050277610, US 20050244858; and U.S. Pat. Nos. 7,297,786, 7,560,438 and 7,056,704, all of which are incorporated herein by reference. Nucleic acid as used herein is understood to include a non-natural polynucleotide (not occurring in nature), for example: a derivative of natural nucleotides such as phosphothionates or peptide nucleic acids (such as those described in the patents and applications cited immediately above). A nucleic acid can be delivered to a cell in order to produce a cellular change that is therapeutic. The delivery of a nucleic acid or other genetic material for therapeutic purposes is gene therapy. The nucleic acid may express a protein or polypeptide, e.g., a protein that is missing or non-functional in the cell or subject. The nucleic acid may be single or double stranded, may be sense or anti-sense, and can be delivered to a cell as naked DNA, in combination with agents to promote nucleic acid uptake into a cell (e.g., transfection reagents), or in the context of a viral vector. The nucleic acid can be targeted to a nucleic acid that is endogenous to the cell (mRNA or microRNA), or a nucleic acid of a pathogen (e.g., viral gene, e.g., hepatitis viral gene). Delivery of a nucleic acid means to transfer a nucleic acid from a container outside a mammal to within the outer cell membrane of a cell in the mammal.

“Obtaining” is understood herein as manufacturing, purchasing, or otherwise coming into possession of.

As used herein, “operably linked” is understood as joined, preferably by a covalent linkage, e.g., joining an amino-terminus of one peptide, e.g., expressing an enzyme, to a carboxy terminus of another peptide, e.g., expressing a signal sequence to target the protein to a specific cellular compartment; joining a promoter sequence with coding or non-coding nucleic acid sequence, in a manner that the two or more components that are operably linked either retain their original activity, or gain an activity upon joining such that the activity of the operably linked portions can be assayed, colocalized, and/or have detectable activity, e.g., enzymatic activity, protein expression activity, nucleic acid levels, etc.

The phrase “pharmaceutically acceptable carrier” is art recognized and includes a pharmaceutically acceptable material, composition or vehicle, suitable for administering compounds of the present invention to mammals. Preferred pharmaceutical carriers for use in the instant invention include those approved for use in humans, particularly organ preservation solutions for use in conjunction with transplants, particularly liver transplants. For example, a preferred carrier for nucleic acid delivery is histidine-tryptophan-ketoglutarate (HTK) solution (6 mM Potassium chloride; 4 mM Magnesium chloride; 0.015 mM Calcium chloride.2H₂O; 18 mM Histidine hydrochloride.H₂O; 180 mM Histidine (mmol/L); 2 mM Tryptophan; 1 mM Potassium hydrogen 2-ketoglutarate; 15 mM Sodium chloride; 30 mM Mannitol; Osmolarity (mOsM) 310; pH 7.4. Another preferred pharmaceutical carrier for use in the methods of the invention includes University of Wisconsin solution (30 mM Raffinose; 100 mM Lactobionate; 5 mM Adenosine; 25 mM Potassium phosphate; 1 mM Allopurinol; 3 mM Glutathione; 10 mM Potassium hydroxide; 5 mM Magnesium sulfate; 27 mM Sodium chloride; 50 g/L Hydroxylethyl starch; 40 U/L Insulin; 16 mg/L Dexamethasone; 200,000 U/L Penicillin; Osmolarity (mOsM) 320; pH 7.4). It is understood that nucleic acids can be resuspended in another solution, e.g., normal saline, sterile water, and added to the organ preservation solution. The carriers include liquid diluent, excipient, solvent involved in carrying or transporting the subject agent from outside of the liver, to inside a cell in the liver.

It is understood that other agents may and will likely be delivered in conjunction with a liver transplant including, but not limited to, immunosuppressive agents, antibiotics, analgesics, etc. The carrier for delivery of each of these agents can be selected separately. Each carrier must be “acceptable” in the sense of being compatible with the other ingredients of the formulation and not injurious to the patient. For example, pharmaceutically acceptable carriers for administration of cells typically is a carrier acceptable for delivery by injection, and do not include agents such as detergents or other compounds that could damage the cells to be delivered. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations, particularly phosphate buffered saline solutions which are preferred for intraocular delivery.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

Formulations of the present invention include those suitable for intraheptaic, oral, nasal, topical, transdermal, buccal, sublingual, intramuscular, intraperotineal, intraocular, intraheptaic, and/or other routes of parenteral administration. The specific route of administration will depend, inter alia, on the specific cell to be targeted. The formulations may conveniently be presented in unit dosage form and may be prepared by any methods well known in the art of pharmacy. The amount of active ingredient that can be combined with a carrier material to produce a single dosage form will generally be that amount of the compound that produces a therapeutic effect.

“Plasmid DNA” is understood as a closed circular DNA extra chromosomal DNA molecule separate from the chromosomal DNA which is capable of replicating independently from the chromosomal DNA in at least one organism. Plasmid DNA for use in the method of the invention can be produced, for example, in a bacteria, a yeast, an insect cell, a mammalian cell, or any other type of cell. The cell type in which the plasmid can be replicated is dependent upon the regulatory sequences provided therein. Plasmid DNA can vary in size from about 1 kb to about 1000 kb. However, in the context of the instant invention, a plasmid is typically about 5 kb to 20 kb. Plasmids can be supercoiled DNA and do not typically contain any proteins (e.g., histones) to promote organization of the nucleic acid. It is understood that plasmid DNA can be cut, for example, with restriction enzymes to provide linear DNA molecules.

As used herein, “plurality” is understood to mean more than one. For example, a plurality refers to at least two, three, four, five, or more.

A “polypeptide” or “peptide” as used herein is understood as two or more independently selected natural or non-natural amino acids joined by a covalent bond (e.g., a peptide bond). A peptide can include 2, 3, 4, 5, 6, 7, 8, 9, 10, 11, 12, 13, 14, 15, 16, 17, 18, 19, 20, or more natural or non-natural amino acids joined by peptide bonds. Polypeptides as described herein include full length proteins (e.g., fully processed proteins) as well as shorter amino acids sequences (e.g., fragments of naturally occurring proteins or synthetic polypeptide fragments).

As used herein, “prevention” is understood as to limit, reduce the rate or degree of onset, or inhibit the development of at least one sign or symptom of a disease or condition particularly in a subject prone to developing the disease or disorder. For example, a subject undergoing liver transplant due to liver damage as a result of hepatitis, particularly hepatitis C infection will not develop hepatitis C infection of the transplanted liver for at least 0.25 years, 0.5 years, 1 year, 1.5 years, 2 years, 3 years, 4 years, 5 years, 6 years, 7 years, 8 years, 9 years, or 10 year after transplantation of the new liver. A subject undergoing liver transplantation due to liver damage will not suffer from liver failure for at least 1 year, 1.5 years, 2 years, 3 years, 4 years, 5 years, 6 years, 7 years, 8 years, 9 years, or 10 year after transplantation of the new liver. Prevention can require the administration of more than one dose of an agent or therapeutic.

A “sample” as used herein refers to a biological material that is isolated from its environment (e.g., blood or tissue from an animal, cells, or conditioned media from tissue culture) and is suspected of containing, or known to contain an analyte, such as a virus, an antibody, or a product from a reporter construct. A sample can also be a partially purified fraction of a tissue or bodily fluid. A reference sample can be a “normal” sample, from a donor not having the disease or condition fluid, or from a normal tissue in a subject having the disease or condition (e.g., liver cells from a subject not infected with hepatitis C, non-liver cells from a subject infected with hepatitis C). A reference sample can also be from an untreated donor or cell culture not treated with an active agent (e.g., no treatment or administration of vehicle only). A reference sample can also be taken at a “zero time point” prior to contacting the cell or subject with the agent or therapeutic intervention to be tested.

“Small molecule” as used herein is understood as a compound, typically an organic compound, having a molecular weight of no more than about 1500 Da, 1000 Da, 750 Da, or 500 Da, 250 Da, 100 Da; or any molecular weight bracketed by those values. In an embodiment, a small molecule does not include a polypeptide or nucleic acid.

A “subject” as used herein refers to living organisms. In certain embodiments, the living organism is an animal. In certain preferred embodiments, the subject is a mammal. In certain embodiments, the subject is a domesticated mammal or a primate including a non-human primate. Examples of subjects include humans, monkeys, dogs, cats, mice, rats, cows, horses, goats, and sheep. A human subject may also be referred to as a patient.

A subject “suffering from or suspected of suffering from” a specific disease, condition, or syndrome has a sufficient number of risk factors or presents with a sufficient number or combination of signs or symptoms of the disease, condition, or syndrome such that a competent individual would diagnose or suspect that the subject was suffering from the disease, condition, or syndrome. Methods for identification of subjects suffering from or suspected of suffering from hepatitis, e.g., hepatitis C is within the ability of those in the art. Subjects suffering from, and suspected of suffering from, a specific disease, condition, or syndrome are not necessarily two distinct groups.

As used herein, “susceptible to” or “prone to” or “predisposed to” a specific disease or condition and the like refers to an individual who based on genetic, environmental, health, and/or other risk factors is more likely to develop a disease or condition than the general population. An increase in likelihood of developing a disease may be an increase of about 10%, 20%, 50%, 100%, 150%, 200%, or more.

“Therapeutically effective amount,” as used herein refers to an amount of an agent which is effective, upon single or multiple dose administration to the cell or subject, in prolonging the survivability of the patient with such a disorder, reducing one or more signs or symptoms of the disorder, preventing or delaying and the like beyond that expected in the absence of such treatment.

An agent or other therapeutic intervention can be administered to a subject, either alone or in combination with one or more additional therapeutic agents or interventions, as a pharmaceutical composition in mixture with conventional excipient, e.g., pharmaceutically acceptable carrier, or therapeutic treatments.

The pharmaceutical agents may be conveniently administered in unit dosage form and may be prepared by any of the methods well known in the pharmaceutical arts, e.g., as described in Remington's Pharmaceutical Sciences (Mack Pub. Co., Easton, Pa., 21^{st} Edition, 2006). Formulations for parenteral administration may contain as common excipients such as sterile water or saline, polyalkylene glycols such as polyethylene glycol, oils of vegetable origin, hydrogenated naphthalenes and the like. In particular, biocompatible, biodegradable lactide polymer, lactide/glycolide copolymer, or polyoxyethylene-polyoxypropylene copolymers may be useful excipients to control the release of certain agents.

It will be appreciated that the actual preferred amounts of active compounds used in a given therapy will vary according to e.g., the specific compound being utilized, the particular composition formulated, the mode of administration and characteristics of the subject, e.g., the species, sex, weight, general health and age of the subject, the route of administration, e.g., in vivo, ex vivo. Optimal administration rates for a given protocol of administration can be readily ascertained by those skilled in the art using conventional dosage determination tests conducted with regard to the foregoing guidelines.

Ranges provided herein are understood to be shorthand for all of the values within the range. This includes all individual sequences when a range of SEQ ID NOs: is provided. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Unless specifically stated or obvious from context, as used herein, the term “or” is understood to be inclusive.

Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Nucleic acids encoding the various polypeptide sequences can readily be determined by one of skill in the art, and any sequence encoding any of the polypeptide sequences of the invention falls within the scope of the invention.

Any compositions or methods provided herein can be combined with one or more of any of the other compositions and methods provided herein.

As used herein, the compounds of this invention are defined to include pharmaceutically acceptable derivatives thereof. A “pharmaceutically acceptable derivative” means any pharmaceutically acceptable salt, ester, salt of an ester, or other derivative of a compound of this invention which, upon administration to a recipient, is capable of providing (directly or indirectly) a compound of this invention. Particularly favored derivatives are those that increase the bioavailability of the compounds of this invention when such compounds are administered to a mammal (e.g., by allowing an orally administered compound to be more readily absorbed into the blood, to increase serum stability or decrease clearance rate of the compound) or which enhance delivery of the parent compound to a biological compartment (e.g., the brain or lymphatic system) relative to the parent species. Derivatives include derivatives where a group which enhances aqueous solubility or active transport through the gut membrane is appended to the structure of formulae described herein.

The compounds of this invention may be modified by appending appropriate functionalities to enhance selective biological properties. Such modifications are known in the art and include those which increase biological penetration into a given biological compartment (e.g., blood, lymphatic system, central nervous system), increase oral availability, increase solubility to allow administration by injection, alter metabolism and alter rate of excretion. Pharmaceutically acceptable salts of the compounds of this invention include those derived from pharmaceutically acceptable inorganic and organic acids and bases. Examples of suitable acid salts include acetate, adipate, benzoate, benzenesulfonate, butyrate, citrate, digluconate, dodecylsulfate, formate, fumarate, glycolate, hemisulfate, heptanoate, hexanoate, hydrochloride, hydrobromide, hydroiodide, lactate, maleate, malonate, methanesulfonate, 2-naphthalenesulfonate, nicotinate, nitrate, palmoate, phosphate, picrate, pivalate, propionate, salicylate, succinate, sulfate, tartrate, tosylate and undecanoate. Salts derived from appropriate bases include alkali metal (e.g., sodium), alkaline earth metal (e.g., magnesium), ammonium and N-(alkyl)₄₊ salts. This invention also envisions the quaternization of any basic nitrogen-containing groups of the compounds disclosed herein. Water or oil-soluble or dispersible products may be obtained by such quaternization.

The compounds of the invention can, for example, be administered ex vivo by injection, intraheptatically, with a dosage ranging from about 0.001 to about 100 mg/kg of body weight, or according to the requirements of the particular drug and more preferably from 0.5-10 mg/kg of body weight (to which the liver will be implanted). For delivery of a small nucleic acid therapeutic (e.g., about 12-50 nucleotides in length, single or double stranded) to a liver ex vivo, effective dosages would range from about 25 mg to about 1000 mg, that is about 50 mg to 500 mg, about 100 mg to about 1000 mg, about 500 mg to about 1000 mg, about 250 mg to about 750 mg, or any range bracketed by any of the two values listed, for an adult liver. For larger nucleic acid therapeutics (e.g., plasmid vectors typically about 5 kb to 20 kb), effective dosages would range from about 25 mg to about 1000 mg, that is about 50 mg to 500 mg, about 100 mg to about 1000 mg, about 500 mg to about 1000 mg, about 250 mg to about 750 mg, or any range bracketed by any of the two values listed, for an adult liver. Dosages can be adjusted for the size of the plasmid delivered.

For administration of viral particles ex vivo, dosages are typically provided by number of virus particles (or viral genomes) and effective dosages would range from about 1×10¹⁰ to 1×10¹⁴ particles, about 1×10¹⁰ to 1×10¹³ particles, about 1×10¹¹ to 1×10¹⁴ particles, about 1×10¹² to 1×10¹⁴ particles, or about 1×10⁹ to 1×10¹⁵ particles delivered to an adult liver (about 1.5 kg). In pediatric cases, dosage can be reduced to correspond to the size of the liver or liver portion being transplanted into the recipient. The effective dose can be the number of particles delivered for each expression construct to be delivered when different expression constructs encoding different genes are administered separately. In alternative embodiment, the effective dose can be the total number of particles administered, of one or more types. The methods herein contemplate administration of an effective amount of compound or compound composition to achieve the desired or stated effect.

The ex vivo methods provided herein overcome the technical difficulties associated with the production of sufficient quantities of viral vectors, particularly AAV viral vectors, to allow for delivery of a therapeutic dose. Similarly, the methods substantially reduce the cost of production of biological therapeutic agents by reducing the amount of agent required. The nucleic acid is delivered only to the target tissue and not diluted in systemic circulation.

Frequency of dosing will depend on the agent administered, the progression of the disease or condition in the subject, and other considerations known to those of skill in the art. For ex vivo administration, administration will typically occur only once, however multiple administrations (e.g., 2, 3, 4, 5, 6, 7, 8 administrations) are possible with subsequent in vivo administration. In the context of the instant invention, recirculation of the agents of the invention through the liver ex vivo is considered administration of a single dose, unless additional compounds of the invention are added to the perfusate. For example, pharmacokinetic and pharmacodynamic considerations for compositions delivered ex vivo and to cold tissue, are different than for systemic administration in vivo e.g., clearance is reduced. Therefore, dosing can be lower with ex vivo administration as compared to in vivo administration. If systemic administration of agents related to immunesupression after transplantation, anti-inflammatory agents, narcotics for pain control, etc., it is expected that the dosing frequency of the other agents will be higher than the siRNA or expression construct, e.g., one or more times daily, one or more times weekly. Dosing may be determined in conjunction with monitoring of one or more signs or symptoms of the disease, e.g., liver function by liver enzyme levels, etc.

The amount of active ingredient that may be combined with the carrier materials to produce a single dosage form will vary depending upon the host treated and the particular mode of administration. A typical preparation will contain from about 1% to about 95% active compound (w/w). Alternatively, such preparations contain from about 20% to about 80% active compound.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, condition or symptoms, method of administration, and the judgment of the treating physician.

Upon improvement of a patient's condition or for prevention of infection or organ rejection, a maintenance dose of a compound, composition or combination of this invention may be administered, if necessary. Subsequently, the dosage or frequency of administration, or both, may be reduced, as a function of the symptoms, to a level at which the improved condition is retained. Patients may, however, require intermittent treatment on a long-term basis upon any recurrence of disease symptoms (e.g. reduced expression from expression construct).

The term “pharmaceutically acceptable carrier” refers to a carrier that can be administered to a patient, together with a compound of this invention, and which does not destroy the pharmacological activity thereof and is nontoxic when administered in doses sufficient to deliver a therapeutic amount of the compound.

The pharmaceutical compositions of this invention may be administered enterally for example intrahepatically, by oral administration, parenterally, intraocularly, by inhalation spray, topically, nasally, buccally, or via an implanted reservoir, preferably by oral or vaginal administration or administration by injection. The pharmaceutical compositions of this invention may contain any conventional non-toxic pharmaceutically-acceptable carriers, adjuvants or vehicles. In some cases, the pH of the formulation may be adjusted with pharmaceutically acceptable acids, bases, or buffers to enhance the stability of the formulated compound or its delivery form. The term parenteral as used herein includes intrahepatic, intraocular, subcutaneous, intracutaneous, intravenous, intramuscular, intraarticular, intraarterial, intrasynovial, intrasternal, intrathecal, intralesional, and intracranial injection or infusion techniques.

The pharmaceutical carriers may be in the form of a sterile injectable preparation, for example, as a sterile injectable aqueous or oleaginous suspension. Preferably agents of the invention are prepared as pharmaceutical compositions in solution acceptable for use in conjunction with liver transplantation in humans, for example, HTK or Wisconsin transplant solution. Pharmaceutically acceptable compositions may be formulated according to techniques known in the art using suitable dispersing or wetting agents (such as, for example, TWEEN® 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as TWEENs® or SPANs® and/or other similar emulsifying agents or bioavailability enhancers which are commonly used in the manufacture of pharmaceutically acceptable solid, liquid, or other dosage forms may also be used for the purposes of formulation.

When the compositions of this invention comprise a combination of a compound of the formulae described herein and one or more additional therapeutic or prophylactic agents, both the compound and the additional agent should be present at dosage levels of between about 1 to 100%, and more preferably between about 5 to 95% of the dosage normally administered in a monotherapy regimen. The additional agents may be administered separately, as part of a multiple dose regimen, from the compounds of this invention. Alternatively, those agents may be part of a single dosage form, mixed together with the compounds of this invention in a single composition.

Effective dosages of the expression constructs of the invention to be administered may be determined through procedures well known to those in the art which address such parameters as biological half-life, bioavailability, route of administration, and toxicity.

### Gene Delivery

Compositions and methods for gene delivery to various organs and cell types in the body are known to those of skill in the art. Such compositions and methods are provided, for example in U.S. Pat. Nos. 7,459,153; 7,282,199; 7,259,151; 7,041,284; 6,849,454; 6,410,011; 6,027,721; and 5,705,151, all of which are incorporated herein by reference. Expression constructs provided in the listed patents and any other known expression constructs for gene delivery can be used in the compositions and methods of the invention.

The viral vectors used in each of the studies demonstrate that various gene therapy viral vector designs can be useful for gene delivery. Methods of viral vector design and generation are well known to those of skill in the art, and methods of preparation of viral vectors can be performed by any of a number of companies as demonstrated below. Expression constructs provided herein can be inserted into any of the exemplary viral vectors listed below. Alternatively, viral vectors can be generated base on the examples provided below.

Gene transfer and nucleic acid therapeutics have been demonstrated to typically be more effective when delivered to the desired site of action rather than systemically, e.g, by delivering the viral vector or siRNA ex vivo, the viral vector or siRNA is delivered to the site of action rather than systemically, both increasing delivery and transduction efficiency and reducing undesirable systemic effects.

Gene transfer to the liver using AAV vectors for the treatment of hemophilia B is currently being tested in a phase 1 trial, see, e.g., clinicaltrials.gov identifier NCT00515710. The study includes intra-hepatic administration of AAV2-hFIX (Factor IX) and secondary outcomes for analysis include determining the potential efficacy in each dose group by measuring biological and physiological activity of the transgene product. This human trial follows a large number of animal experiments in which AAV vectors were efficiently delivered to the liver using AAV2 and AAV8 viral vectors (e.g., Mount et al. Blood. 2002; 99:2670-2676; Cardone et al., Hum Mol Genet. 2006; 15:1225-1236; Daly et al., Gene Ther. 2001; 8:1291-1298; McEachern et al. J Gene Med. 2006; 8:719-729; Koeberl et al., Gene Ther. 2006; 13:1281-1289; Moscioni et al., Mol Ther. 2006; 14:25-33; Park et al., Exp Mol Med. 2006; 38:652-661; Scallan et al. Blood. 2003; 102:2031-2037; Seppen et al. Mol Ther. 2006; 13:1085-1092; each of which is incorporated by reference)

Many studies have demonstrated that local administration to the eye provides efficient transduction of cells with viral vectors. In the Bainbridge study (NEJM, 358:2231-2239, 2008, incorporated herein by reference), the tgAAG76 vector, a recombinant adeno-associated virus vector of serotype 2 was used for gene delivery. The vector contains the human RPE65 coding sequence driven by a 1400-bp fragment of the human RPE65 promoter and terminated by the bovine growth hormone polyadenylation site, as described elsewhere.

Maguire (NEJM, 358:2240-8. 2008, incorporated herein by reference) used the recombinant AAV2.hRPE65v2 viral vector which is a replication-deficient AAV vector containing RPE65 cDNA that has been documented to provide long-term, sustained (>7.5 years, with ongoing observation) restoration of visual function in a canine model of LCA2 after a single subretinal injection of AAV2.RPE65. The cis-plasmid used to generate AAV2.RPE65 contains the kanamycin-resistance gene, and the transgene expression cassette contains a hybrid chicken β-actin (CBA) promoter comprising the cytomegalovirus immediate early enhancer (0.36 kb), the proximal CBA promoter (0.28 kb), and CBA exon 1 flanked by intron 1 sequences (0.997 kb). To include a Kozak consensus sequence at the translational start site, the sequence surrounding the initiation codon was modified.

The viral vector used by Hauswirth (Hum Gene Ther. 19:979-90, 2008, incorporated herein by reference) was a recombinant adeno-associated virus serotype 2 (rAAV2) vector, altered to carry the human RPE65 gene (rAAV2-CB^{SB}-hRPE65), which had been previously demonstrated to restore vision in animal models with RPE65 deficiency. The viral vector includes, in order from 5′ to 3′, an inverted terminal repeat sequence (ITR), a CMV immediate early enhancer, a β-actin promoter, β-actin exon 1, β-actin intron 1, β-actin exon 3, wild-type human RPE65 sequence, SV40 poly(A) sequence, and an inverted terminal repeat. The RPE65-LCA viral vector was delivered by subretinal injection (5.96×10¹⁰ vector genomes in 150 μl).

Additional AAV vectors are provided in the review by Rolling 2004 (Gene Therapy 11: S26-S32, incorporated herein by reference). Hybrid AAV viral vectors, including AAV 2/4 and AAV2/5 vectors are provided, for example, by U.S. Pat. No. 7,172,893 (incorporated herein by reference). Such hybrid virus particles include a parvovirus capsid and a nucleic acid having at least one adeno-associated virus (AAV) serotype 2 inverted terminal repeat packaged in the parvovirus capsid. However, the serotypes of the AAV capsid and said at least one of the AAV inverted terminal repeat are different. For example, a hybrid AAV2/5 virus in which a recombinant AAV2 genome (with AAV2 ITRs) is packaged within a AAV Type 5 capsid.

Self-complementary AAV (scAAV) vectors have been developed to circumvent rate-limiting second-strand synthesis in single-stranded AAV vector genomes and to facilitate robust transgene expression at a minimal dose (Yokoi, 2007. IOVS. 48:3324-3328, incorporated herein by reference). Self-complementary AAV-vectors were demonstrated to provide almost immediate and robust expression of the reporter gene inserted in the vector. Subretinal injection of 5×10⁸ viral particles (vp) of scAAV.CMV-GFP resulted in green fluorescent protein (GFP) expression in almost all retinal pigment epithelial (RPE) cells within the area of the small detachment caused by the injection by 3 days and strong, diffuse expression by 7 days. Expression was strong in all retinal cell layers by days 14 and 28. In contrast, 3 days after subretinal injection of 5×10⁸ vp of single stranded (ss)AAV.CMV-GFP, GFP expression was detectable in few RPE cells. Moreover, the ssAAV vector required 14 days for the attainment of expression levels comparable to those observed using scAAV at day 3. Expression in photoreceptors was not detectable until day 28 using the ssAAV vector. The use of the scAAV vector in the gene delivery methods of the invention can allow for prompt and robust expression from the expression construct. Moreover, the higher level of expression from the scAAV viral vectors can allow for delivery to of the viral particles intravitreally rather than subretinally.

Various recombinant AAV viral vectors have been designed including one or more mutations in capsid proteins or other viral proteins. It is understood that the use of such modified AAV viral vectors falls within the scope of the instant invention.

Kota et al. (Cell, 137: 1005-1017, 2009, incorporated herein by reference) demonstrated efficient delivery of an AAV expression vector containing an shRNA targeted to miR-26a in vivo in a model of rat hepatocellular carcinoma.

Adenoviral vectors have also been demonstrated to be useful for gene delivery. For example, Mori et al (2002. IOVS, 43:1610-1615, incorporated herein by reference) discloses the use of an adenoviral vector that is an E-1 deleted, partially E-3 deleted type 5 Ad in which the transgene (green fluorescent protein) is driven by a CMV promoter. Peak expression levels were demonstrated upon injection of 10⁷ to 10⁸ viral particles, with subretinal injection providing higher levels of expression than intravitreal injection.

Efficient non-viral ocular gene transfer was demonstrated by Farjo et al. (2006, PLoS 1:e38, incorporated herein by reference) who used compacted DNA nanoparticles as a system for non-viral gene transfer to ocular tissues. As a proof of concept, the pZEEGFP5.1 (5,147 bp) expression construct that encodes the enhanced green fluorescent protein (GFP) cDNA transcriptionally-controlled by the CMV immediate-early promoter and enhancer was used. DNA nanoparticles were formulated by mixing plasmid DNA with CK30PEG10K, a 30-mer lysine peptide with an N-terminal cysteine that is conjugated via a maleimide linkage to 10 kDa polyethylene glycol using known methods. Nanoparticles were concentrated up to 4 mg/ml of DNA in saline. The compacted DNA was delivered at a 0.6 μg dose to the vitreal cavity. GFP expression was observed in the lens, retina, and pigment epithelium/choroid/sclera by PCR and microscopy.

Grimm and Kay (J Clin Invest. 117: 3633-3641, 2007, incorporated herein by reference) provide an overview of therapeutic siRNAs and their delivery as duplexed RNA or in an expression vector for the expression of an shRNA.

Further, a number of patents have been issued for methods of ocular gene transfer including, but not limited to, U.S. Pat. No. 7,144,870 which provides methods of hyaluronic acid mediated adenoviral transduction; U.S. Pat. Nos. 7,122,181 and 6,555,107 which provide lentiviral vectors and their use to mediate ocular gene delivery; U.S. Pat. No. 6,106,826 which provides herpes simplex viral vectors and their use to mediate ocular gene delivery; and U.S. Pat. No. 5,770,580 which provides DNA expression vectors and their use to mediate ocular gene delivery. Each of these patents is incorporated herein by reference.

Hepatic gene delivery has also been demonstrated in a number of studies. For example, self-complementary adeno-associated virus vectors containing a novel liver-specific human factor IX expression cassette were found to enable highly efficient transduction of murine and nonhuman primate liver (Nathwani et al., Blood. 2006 Apr. 1; 107:2653-61). An AAV-2 genome encoding the hfIX gene was cross-packaged into capsids of AAV types 1 to 6 using efficient, large-scale technology for particle production and purification. In immunocompetent mice, the resultant vector particles expressed high hFIX levels ranging from 36% (AAV-4) to more than 2000% of normal (AAV-1, -2, and -6), which would exceed curative levels in patients with hemophilia. (Grimm et al., Blood. 2003 Oct. 1; 102:2412-9).

Further, a number of patents have been issued for methods of hepatic gene or nucleic acid transfer including, but not limited to U.S. Pat. Nos. 7,615,537 and 7,351,813 which provide methods for expression of clottings factor in the liver; U.S. Pat. No. 7,528,118 which provides methods for delivery of siRNA to liver to reduce expression of ApoB; U.S. Pat. No. 7,498,017 provides an ationic poly cyclic imidazolinium-containing compound for condensing nucleic acid for delivery to a cell, including a liver cell; and U.S. Pat. No. 6,967,018 for delivery of AAV-1, 2, or 5 vectors for the expression of adiponectin. Each of these patents related to hepatic gene or nucleic acid transfer is incorporated herein by reference.

### Self-Complementary Adenoviral Vectors

Under normal circumstances, AAV packages a single-stranded DNA molecule of up to 4800 nucleotides in length. Following infection of cells by the virus, the intrinsic molecular machinery of the cell is required for conversion of single-stranded DNA into double stranded form. The double-stranded form is then capable of being transcribed, thereby allowing expression of the delivered gene to commence. It has been shown in a number of cell and tissue types that second strand synthesis of DNA by the host cell is the rate-limiting step in expression. By virtue of already being packaged as a double stranded DNA molecule, self-complementary AAV (scAAV) bypasses this step, thereby greatly reducing the time to onset of gene expression.

Self-complementary AAV is generated through the use of vector plasmid with a mutation in one of the terminal resolution sequences of the AAV virus. This mutation leads to the packaging of a self-complementary, double-stranded DNA molecule covalently linked at one end. Vector genomes are required to be approximately half genome size (2.4 KB) in order to package effectively in the normal AAV capsid. Because of this size limitation, large promoters are unsuitable for use with scAAV. Most broad applications to date have used the cytomegalovirus immediate early promoter (CMV) alone for driving transgene expression. A long acting, ubiquitous promoter of small size is useful in a scAAV system.

Xu et al (Mol. Ther. 11: 523-530, 2005, incorporated herein by reference) have demonstrated efficient shRNA expression in mammalian cancer cells after delivery using an scAAV vector. U.S. Pat. No. 7,465,583 teaches delivery of nucleic acid to various cell types using scAAV vectors (incorporated herein by reference).

### Gene Transfer Using Plasmid DNA

Delivery of plasmid DNA has been demonstrated to be an efficient method of gene transfer in vivo (Yoshino et al., 2006, and Budker et al, 1996; Zhang et al., 1997; each incorporated herein by reference). The methods provided herein are directed to gene transfer ex vivo. In the methods, the nucleic acid is delivered directly to the tissue. Therefore, the nucleic acid need not be packaged or modified to direct it to the appropriate tissue. Moreover, as the nucleic acid is delivered to the cells over a short time period, e.g., about 1 hour or less, and the liver is typically cold, the nucleic acid is far less susceptible to the effects of nucleases than a nucleic acid delivered systemically. The invention provides expression of a coding sequence in naked DNA which includes DNA not enclosed in a viral capsid, but can include other compounds to promote cellular uptake and/or to increase the stability of the DNA. Such compounds are preferably safe for use in humans and such considerations are well known to those of skill in the art. Typically, naked DNA is in the form of plasmid DNA, such as supercoiled plasmid DNA to provide some protection against nucleases that may be present. Gene transfer using plasmid DNA can also include the use of plasmid DNA that has been cut, for example, with a restriction enzyme, to provide a linear DNA molecule. Gene transfer using plasmid DNA may be beneficial as it may overcome the obstacle of immune response to viral capsid proteins.

### Nucleic Acid Regulatory Sequences

The invention provides expression constructs that include any regulatory sequences that are functional in the cells in which shRNA expression is desired, e.g., liver cells. For example, cell and tissue specific promoters such as the thyroid hormone-binding globulin promoter, alpha-1-antitrypsin promoter, and LP-1 promoter. Alternatively, non-tissue specific promoters including viral promoters such as cytomegalovirus (CMV) promoter, β-actin promoter including the chicken β-actin promoter, phosphoglycerate kinase (PGK) promoter, ubiquitin promoter including hybrid ubiquitin promoter, and EF-1α promoter can be used.

The chimeric CMV-chicken β-actin promoter (CBA) has been utilized extensively as a promoter that supports expression in a wide variety of cells in rAAV vectors. In addition to broad tropism, CBA also has the capacity to promote expression for long periods post infection (Acland, G. M. et al. MoI Then, 2005, 12:1072-1082, incorporated herein by reference). CBA is about 1700 base pairs in length, too large in most cases to be used in conjunction with scAAV to deliver cDNAs (over 300 bps pairs in length), however it can be used to deliver siRNA and shRNAs. CBA is a ubiquitous strong promoter composed of a cytomegalovirus (CMV) immediate-early enhancer (381 bp) and a CBA promoter-exon1-intron1 element (1,352 bp) (Raisler Proc Natl Acad Sci USA. 99: 8909-8914, 2002, incorporated herein by reference). A shortened CBA promoter sequence, the smCBA promoter sequence, has also been described. The total size of smCBA is 953 bps versus 1714 bps for full length CBA. The smCBA promoter is described in Mah, et al. 2003 (Hum. Gene Ther. 14:143-152, incorporated herein by reference) and Haire, et al. 2006 (IOVS, 2006, 47:3745-3753, incorporated herein by reference). Ubiquitin promoters, including hybrid ubiquitin promoters, are provided, for example, in U.S. Pat. No. 7,452,716.

Other regulatory sequences for inclusion in expression constructs include poly-A signal sequences, for example SV40 polyA signal sequences. The inclusion of a splice site (i.e., exon flanked by two introns) has been demonstrated to be useful to increase gene expression of proteins from expression constructs.

Enhancers can also be used in the constructs of the invention. Enhancers include, but are not limited to enhancer is selected from the group consisting of cytomegalovirus (CMV) enhancer, an elongation factor 1-alpha enhancer, and liver-specific enhancers.

For viral sequences, the use of viral sequences including inverted terminal repeats, for example in AAV viral vectors can be useful. Certain viral genes can also be useful to assist the virus in evading the immune response after administration to the subject.

In certain embodiments of the invention, the viral vectors used are replication deficient, but contain some of the viral coding sequences to allow for replication of the virus in appropriate cell lines. The specific viral genes to be partially or fully deleted from the viral coding sequence is a matter of choice, as is the specific cell line in which the virus is propagated. Such considerations are well known to those of skill in the art.

Further, viruses with specific tropisms that will cause them to go to efficiently infect liver cells can be selected for use in the method of the invention. For example, the AAV8 serotype is known to be preferentially hepatotrophic (Nakai et al., 2005. J. Virol. 79:214-224).

### Kits

The present invention also encompasses a finished packaged and labeled pharmaceutical product or laboratory reagent. This article of manufacture includes the appropriate instructions for use in an appropriate vessel or container such as a glass vial or other container that is hermetically sealed. A pharmaceutical product may contain, for example, a compound of the invention in a unit dosage form in a first container, and in a second container, sterile water or adjuvant for injection. The unit dosage form may be a solid suitable for ex vivo delivery.

As with any pharmaceutical product, the packaging material and container are designed to protect the stability of the product during storage and shipment. Further, the products of the invention include instructions for use or other informational material that advise the physician, technician, or patient on how to appropriately prevent or treat the disease or disorder in question. In other words, the article of manufacture includes instructions indicating or suggesting a dosing regimen including, but not limited to, actual doses, monitoring procedures (e.g. liver enzyme level testing), and other monitoring information.

Specifically, the invention provides an article of manufacture including packaging material, such as a box, bottle, tube, vial, container, sprayer, needle for intrahepatic administration, envelope and the like; and at least one unit dosage form of a pharmaceutical agent contained within said packaging material, wherein said pharmaceutical agent comprises a compound of the invention, and wherein said packaging material includes instruction which indicate that said compound can be used to prevent, manage, treat, and/or ameliorate one or more symptoms associated with hepatitis, particularly hepatitis C, or liver transplant using specific dosing regimens as described herein.

### Co-Administration of Compounds

The compositions and methods of the invention can be combined with any other composition(s) and method(s) known or not yet known in the art for the prevention, amelioration, or treatment of hepatitis, particularly hepatitis C and liver transplantation. It is understood that the treatment of a subject undergoing liver transplantation is a complex process that includes the administration of a number of therapeutic agents as determined by the treating physicians and others of skill in the art. The compositions and methods provided herein can be used in combination with any other therapeutic methods deemed appropriate by the treating physician.

Other strategies for uses of siRNA, shRNA, antisense nucleic acids, and other agents for the treatment of diseases related to hepatitis, particularly hepatitis C, and liver transplantation can be envisioned.

### Antisense Nucleic Acids

Antisense nucleic acids have been designed to hybridize to RNAs, both endogenous cellular RNAs and RNAs present in pathogens, to disrupt the translation from RNAs, inhibiting expression of the gene product, or to downregulate the expression of a microRNA. A number of antisense nucleic acids have been designed for the treatment and prevention of hepatitis C including, but not limited to those included in U.S. Pat. Nos. 6,284,458; 6,391,542, and 6,423,489 all of which are incorporated herein by reference. A number of patents have also been issued that are directed to antisense nucleic acids to inhibit the expression of intracellular adhesion molecule-1 (ICAM-1) including, but not limited to, those provided in U.S. Pat. Nos. 5,883,082 and 6,849,612. Such oligonucleotides are useful, for example, for the preservation of explants ex vivo prior to transplantation, e.g., corneal explants.

Delivery of siRNA or shRNA to Liver Ex Vivo

Inhibition of endogenous liver gene function with siRNA has been studied to reduce hepatic injury, viral infection, or ischemia/reperfusion injury. siRNA could be used in the transplantation setting to reduce ischemia/reperfusion injury, viral recurrence, or tumor recurrence. Previous reports have described siRNA delivery to an explanted liver graft (Inoue et al., Transplantation. 2004; 77:997-1003; Sato, et al. Transplantation 2005; 79:240-243; Tsoulfas et al., J Surg Res 2006; 135:242-249). Sato et al reported delivery of siRNA using a catheter-based in vivo injection method and were able to demonstrate transient down regulation of a target gene in the transplanted liver (Sato et al., Biomaterials 2007; 28:1434-1442). However, Inoue et al reported that several recipients died of graft congestion or thrombus formation even with a relatively selective and small volume injection and they proposed treating the donor several days prior to organ procurement. A potentially more clinically realistic delivery method was proposed by Tsoulfas et al. In their study, they applied ex vivo rapid hydrodynamic injection of pDNA into the preserved liver graft using the hepatic vein. They tested various volumes of delivery to optimize the technique and reported that 50% of the liver weight was an optimal solution volume to balance pDNA delivery and liver injury, a finding similar to optimal conditions reported from in vivo studies.

The hydrodynamic injection technique of siRNA delivery involves intravascular injection of a relatively large amount of physiologic solution along with the nucleic acid molecule. In prior studies, optimal conditions for nucleic acid delivery in vivo were determined to be rapid rather than slow injection, and hepatic vein including tail vein rather than portal vein route (Zhang et al., Hum Gene Ther 1999; 10:1735-1737). The injection volume utilized was equal to approximately 10% of the animal's weight. These seemingly harsh conditions generated acceptable hepatocyte toxicity and were well tolerated in at least one report (Zhang et al., Hum Gene Ther 1999; 10:1735-1737). However, in another study, the authors reported a 60% mortality rate in rats when 10% body weight volume was injected (Inoue S, Transplantation 2004; 77:997-1003). Therefore, systemic hydrodynamic delivery methods require further analysis prior to utilization in the clinical setting. Eastman et al suggested selective injection of a relatively small volume directly into liver using a catheter based method (Eastman et al., Hum Gene Ther 2002; 13:2065-2077) and Inoue et al reported success by decreasing the injection volume to 2.5% of body weight with this method. However, in the liver transplantation setting, an ischemia reperfusion injury aggravated hydrodynamic injection. Inoue et al reported a high incidence of graft failure (graft congestion or venous thrombosis) when the catheter based hydrodynamic procedure was performed on the day of organ harvest.

Another option for gene delivery during liver transplant is ex vivo injection of pDNA into the cold preserved liver graft after procurement. Tsoulfas et al suggested rapid hepatic vein injection with a volume of 50% of the graft weight, which was found to be safe and effective. In our studies, all animals that the received a portal vein injection with a volume of 50% of graft weight survived and showed acceptable ALT level at 24 hours after transplantation. Furthermore, a significant degree of target gene knockdown could be achieved with this technique. In addition, we found that in our ex vivo model, slow injection was superior to conventional rapid injection in terms of functional outcome and injury.

In an ex vivo injection model, cells even minimally injured by hydrodynamic injection may be at high risk due to additional subsequent ischemia-reperfusion injury and the immunological response. In one in vivo study, hepatic vein injection induced highly disrupted cellular architecture in 3-4% of hepatocytes in the mouse liver (Budker et al. J Gene Med 2006; 8:874-888). These apparently dead or dying cells were also observed to contain large amounts of plasmid DNA. Another study utilizing animals co-injected with an enhanced yellow fluorescent protein expression vector and fluorescently labeled immunoglobulin demonstrated hepatocytes flooded with large amounts of IgG which appeared permanently damaged and did not express enhanced yellow fluorescent protein (Sebestyen et al., J Gene Med 2006; 8:852-873).

As demonstrated using the delivery methods herein, some periportal areas with strong fluorescence seemed to overlap with necrotic areas seen after transplantation. Therefore, there is a balance between level of gene delivery and the ability of the transfected cell to maintain integrity and avoid cell death. The balance point should probably be moved towards lower injury direction in the transplantation setting. In fact, one of the reasons for poor functional knockdown in our rapid injection group may be related to higher cell damage caused by the high pressure.

However, lower functional knockdown in hepatocytes in the rapid injection group cannot be fully explained by an injury mechanism according to the histological results. In H&E and TUNEL staining of the transplanted liver at 24 hours after rapid injection, we did not observe massive necrosis in the liver in any cases, even though there were several foci of necrosis and apoptosis. These findings did not support severe damage in the rapid injection group. A dropout theory might explain the difference. In an in vivo study, hydrodynamic injection induced damage would not impair hepatic microcirculation, presumably enabling the clearance of most apoptotic or necrotic hepatocytes by a ‘dropout pathway’ (Budker et al., J Gene Med 2006; 8:874-888.). Dead cells lose attachment to neighboring cells and would exit the liver with hepatic vein flow, thereby not requiring macrophage recruitment and phagocytosis. Similar dropout of damaged cells might take place in an ex vivo model.

The slow injection group in our study showed a 40% transfection rate by morphologic study, however, an average 70% target gene knockdown in hepatocytes was observed. The difference between the morphologic and functional results may be due to cells with only small amounts of fluorescent-labeled siRNA which are not detected by morphologic study but which give functional knockdown. One published study used three consecutive injections within one day to generate approximately 90% target gene knockdown in hepatocytes, but the average level of target gene expression using a single in vivo hydrodynamic injection is typically 40-60% of normal overall liver expression. Previous liver transplantation studies using in vivo hydrodynamic injection one or two days before transplantation showed similar functional knockdown rates. There are many factors that contribute to the rate of functional knockdown observed, however, the functional outcome of the ex vivo slow injection group in this study seemed comparable or superior to those of other in vivo studies using a single hydrodynamic injection.

There are several advantages of an ex vivo injection model. Compared to in vivo injection in which the siRNA containing solution is diluted into the entire blood volume, liver cells have contact with relatively high concentrated nucleic acid containing solution, e.g., siRNA solution or viral solution, until reperfusion in an ex vivo model. Furthermore, there is no flow for a period (one hour in this study) which may facilitate receptor binding or stabilization of injected vesicles in hepatocytes. It has been postulated that blood flow just after hydrodynamic injection may interfere with further stabilization steps. Liu et al. reported the importance of temporary block of blood flow by their clamping study and proposed a hypothesis for gene transfer which suggested that DNA binding is weak and can be easily dissociated by blood flow under normal physiologic conditions (Liu and Huang J Gene Med 2001; 3:569-576.). Other possible advantage is that extracellular nucleases can be excluded in ex vivo model. One of the main barriers to the in vivo use of RNAi is that nucleic acids including siRNA are rapidly degraded by both extracellular and intracellular nucleases.

Nucleases in the blood serve to reduce any nucleic acids contacting the blood including those exogenously administered. The absence of blood in the vasculature in an ex vivo model may help to reduce siRNA degradation

The first hydrodynamic gene delivery into the liver involved the injection of DNA solution into portal vein. However, injection via systemic vein (hepatic vein or tail vein) has been shown to be highly effective and technically easy, so, it has become the more commonly used method of gene delivery to liver. However, in this study using an ex vivo model, there was no difference between the portal vein route and the retrograde route in terms of morphologic delivery area and injury rate. We therefore chose the portal vein route over the IVC to respect normal physiologic flow directions.

As demonstrated herein, siRNA can be delivered into the explanted liver graft using ex vivo hydrodynamic injection with target gene knockdown achieved after transplantation. Slow injection is superior to conventional rapid injection due to reduction of cellular injury and in terms of functional knockdown.

As demonstrated herein, siRNA can be delivered into a cold preserved liver graft using an ex vivo delivery method and that target gene knockdown can be effectively achieved after transplantation.

The following examples are provided merely as illustrative of various aspects of the invention and shall not be construed to limit the invention in any way.

### EXAMPLES

### Example 1

### Materials and Methods

### Animals

Male Lewis rats weighing 175 to 250 g (Harlan Sprague Dawley Inc., Indianapolis, Ind.) were used. Animals were maintained in pathogen-free facility of Johns Hopkins Medical Institutions. Animals were housed and cared for in compliance with the Johns Hopkins University Animal Care and Use Committee.

siRNA

All siRNAs used in this study were obtained from Dharmacon (Lafayette, Colo., USA). In morphologic studies, a modified version of ICAM siRNA (5′-GGAGGUGACUGAGAAGUUG-3′) with a Cy3 molecule added to the 5′ end of each strand was utilized. The siRNA targeting cyclophilin mRNA (ON-TARGETp/us siCONTROL Cyclophilin B siRNA, rat D-001820-50) was used to evaluate functional delivery rate. LacZ-specific siRNAs were used as a control.

siRNA Injection Groups

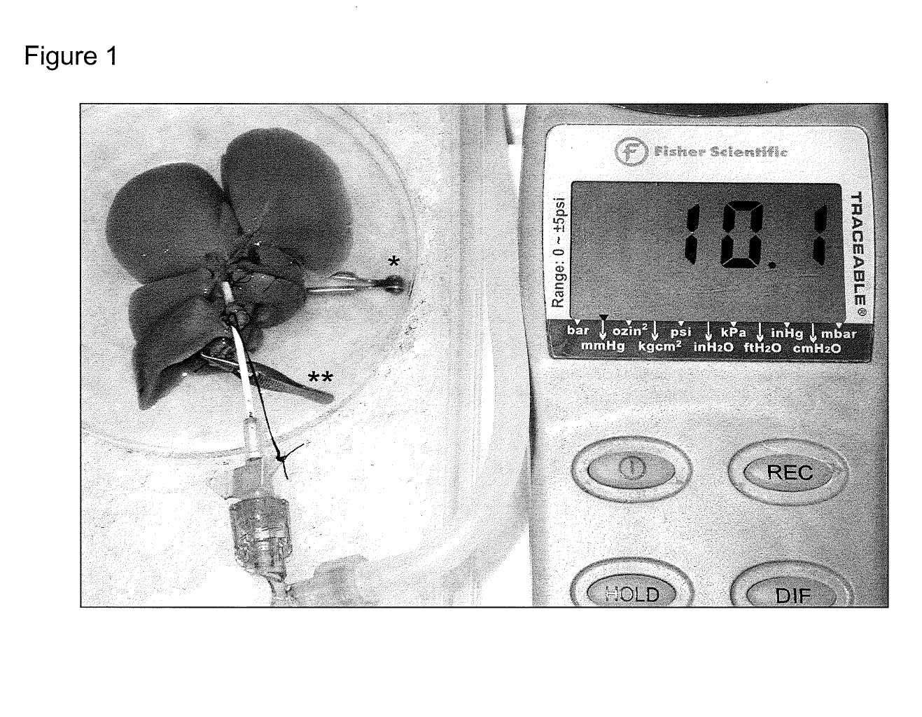

Within 10 minutes of cold preservation, 200 ug of siRNA (2-10 mg/kg of liver) in 6 ml of HTK was injected with various methods. Two different routes (IVC and portal vein (PV)) and two different injection speeds (rapid and slow) were tested. After cannulation of the infrahepatic IVC or PV, the remaining volume was passed slowly, and the suprahepatic and infrahepatic IVC were clamped. A picture from a slow PV-injection is shown in FIG. 1.

In rapid injection group, 50% of liver weight was injected rapidly (over 3-5 seconds). Pressure was measured using a manometer (06-664-18, Fisher Scientific inc., Newark, N.J., USA) during injection. The pressure was measured via a three-way stopcock and went up to 90-100 mm Hg rapidly and decreased to less than 10 mm Hg within 15 seconds. The pressure measured through the outflow vessel (the IVC in the case of injecting through portal vein) went up to 30-40 mm Hg rapidly. Occluding clamps were removed 1 minute later.

In the slow injection group, an identical volume was injected slowly to maintain a pressure between 10 to 13 mm Hg (FIG. 1). The duration of injection was 45 to 90 seconds. After injection, occluding clamps were removed immediately.

### Morphologic Delivery Rate Study

Cy3-labeled siRNAs were injected via the different methods described above. After maintaining the injected livers on ice for 1 hour, a portion of the left, middle, and right lobes were sliced into 5 mm sections and fixed overnight in 4% paraformaldehyde in PBS using routine methods. The tissue sections were placed in 20% sucrose for 4 hours and then frozen in OCT (Sakura Finetek, Torrance, Calif., USA). 5 mm frozen sections were cut and counterstained for 20 min in a 1:400 dilution of Alexa fluor 488 phalloidin (Molecular Probes, Invitrogen®, Carlsbad, Calif., USA). Confocal microscopy was performed on a Zeiss® Axiovert 200M with an LSM 5 Pascal scanning module. Three representative fields were selected from each of the three lobes and the delivery efficacy rate was determined with the image J program (NIH, USA). Percentage of fluorescent positive area was considered as delivery rate. Mean of delivery rate from three different lobes was considered as overall delivery rate.

### Injury Study I (Trypan Blue Uptake Prior to Transplantation)

To measure the degree of injury induced by hydrodynamic injection, 40-50 ml of 200 mmol cold Trypan blue solution was perfused via the portal vein of the cold preserved liver graft at 1 hour after hydrodynamic injection, followed by 30-40 ml of cold 4% paraformaldehyde perfusion. Each lobe was sliced and specimens placed in 4% paraformaldehyde for 20 minutes then slices were frozen in OCT.

5 mm frozen sections were cut and counterstained with Eosin (Sigma-Aldrich, MO, USA). Three representative fields were selected from each of the three lobes and Trypan blue positive cells were counted under the microscope. The mean of the number of positive cells from the three lobes was considered as overall positive number per high power field (HPF, ×200).

### Liver Transplantation

Rats were induced and maintained under general anesthesia using inhaled isoflurane during all procedures. The livers were perfused with cold HTK solution via portal vein, harvested and placed in ice-cold HTK. HTK solution with siRNA was injected within 10 minutes after procurement, the injected livers were maintained at 4° C. for 45 min, and orthotopic liver transplantation was performed using a modification of the previously described cuff technique (Kamada et al., Surgery 1983; 93:64-69.). No immunosuppression was used. Blood was obtained at 24 hrs following reperfusion. Liver tissue was obtained for histologic study or liver cells were isolated after blood collection.

Injury Study II (Serum ALT after Transplantation)

Alanine aminotransferase (ALT) was taken as a mark of hepatocellular injury. Blood samples were taken from the IVC and ALT was measured by Antech Diagnostics (Success, N.Y., USA) through a service provided by the Johns Hopkins University Department of Comparative Medicine.

### Injury Study III (H&E and TUNEL Staining)

Hematoxylin and Eosin (H&E) staining was done and necrotic area was measured. John Wiley & Sons, Inc. TUNEL staining was performed using a commercial kit following the protocol provided by the manufacturer (Roche® Applied Science, Indianapolis, Ind., USA). Nuclei were counterstained with DAPI and Confocal microscopy was performed.

Cell Isolation from Transplanted Livers

Hepatocytes were isolated from transplanted livers using a two-step collagenase perfusion according previously described techniques (Maemura et al. Immunol Cell Biol 2005; 83:336-343.). Cellular RNA was isolated using Trizol® (Invitrogen®, Carlsbad, Calif., USA) according to the manufacturer's instructions. Several samples were inoculated into glass slides to evaluate the hepatocyte preparation purity.

After 1 hour, non-attached cells were rinsed off, and slides fixed with 4% paraformaldehyde. Albumin antibody (MP Biomedicals, Aurora, Ohio, USA) was used in to differentiate hepatocytes. About 85-90% of the isolated cells were positive for albumin by antibody staining.

### RT-PCR

Gene specific primers were designed using Invitrogen® Oligoperfect designer (Invitrogen®, Carlsbad, Calif., USA). Primer sequences used included: Cyclohilin B (forward 5′-CAAGACCTCCTGGCTAGACG-3′, reverse 5′-TTGTGACTGGCTGCTTTCAC-3′), β-actin (forward 5′-GTCGTACCACTGGCATTGTG-3′, reverse 5′-CTCTCAGCTGTGGTGGTGAA-3′). cDNA was generated using the iScript cDNA Synthesis Kit (Bio-Rad®, Hercules, Calif., USA) and realtime PCR was carried out using SYBR® green PCR master mix (Applied Biosystems, Foster City, Calif., USA) on a real time PCR machine (iCycler, Bio-Rad®, Hercules, Calif., USA) with the following reaction conditions: cDNA synthesis, 95° C. 3 minutes (1 cycle), 95° C. 30 seconds (37 cycles), 60° C. 30 seconds (37 cycles), and 72° C. 30 seconds (37 cycles). Realtime data analysis was done using 1Q5 Optical System Software Version 2.0 (Bio-Rad®, Hercules, Calif., USA). Relative expression of Cyclophilin to β-actin mRNA transcripts was quantified using the relative expression software tool (13). LacZ siRNA treated animals using the same delivery method were used as control for each group.

### AAV Construction and Infection

The development of self-complementary AAV (scAAV) vectors and the availability of AAV serotypes for improved transduction of specific target tissues has expanded the usefulness of this virus for therapeutic gene delivery (Wang et al., 2003 Gene Ther. 10:2105-2111). In particular, these advances allow highly efficient transduction of hepatocytes following systemic administration of scAAV8 vectors.

We constructed a scAAV vector system using routine methods to evaluate the therapeutic potential of anti-HCV siRNAs. To allow facile assessment of target tissue transduction, the vector included enhanced green fluorescent protein (eGFP) driven by the ubiquitously expressed elongation factor 1 alpha (EF1α) promoter. Moreover, since miRNAs are frequently embedded within introns of both protein-coding and noncoding primary transcripts, we cloned our anti-HCV siRNAs into the short intron that is part of the EF1α promoter unit, thus allowing simultaneous production of eGFP and anti-HCV siRNAs from a single transcript.

Expression of both the siRNA of interest and eGFP by transient transfection of HeLa cells. Northern blotting demonstrated that the scAAV.eGFP vector produced an equivalent amount of mature shRNA as a control vector in which the miRNA was in an exonic context. Fluorescent microscopy of transfected cells similarly documented equivalent eGFP expression from scAAV.eGFP and a control vector lacking intronic siRNA sequences were then packaged with the AAV8 serotype for in vivo delivery. 1×10¹² vector genomes (vg) per animal were administered with a single tail-vein injection, and liver tissue was harvested three weeks later for analysis of siRNA and eGFP expression.

Fluorescent microscopy documented over 90% transduction of hepatocytes with both vectors. Importantly, it has previously been demonstrated that AAV8-mediated delivery of some short-hairpin RNA (shRNA) constructs induces acute liver toxicity due to competitive inhibition of the miRNA pathway. We observed an absence of any acute inflammation, fibrosis, or overt histologic evidence of toxicity; and maintenance of normal levels of serum markers of liver function (ALT, AST, alkaline phosphatase, and total and direct bilirubin). These data demonstrate that scAAV8 provides an effective, nontoxic means to deliver miRNAs to the liver.

### Statistical Analysis

Data are represented as the mean±SD (S.E. in RT-PCR data). Comparisons between the different methods were performed using the Student's t-test. Differences were considered significant at p<0.05 (two tailed).

### Example 2

### siRNA Delivery Rate and Toxicity According to Injection Route

Livers were harvested and within 10 minutes of cold preservation, Cy3 labeled ICAM siRNA was injected in HTK by various hydrodynamic injections as described above. First, we tested two different injection routes (inferior vena cava (IVC) and PV) of siRNAs using the rapid injection method.

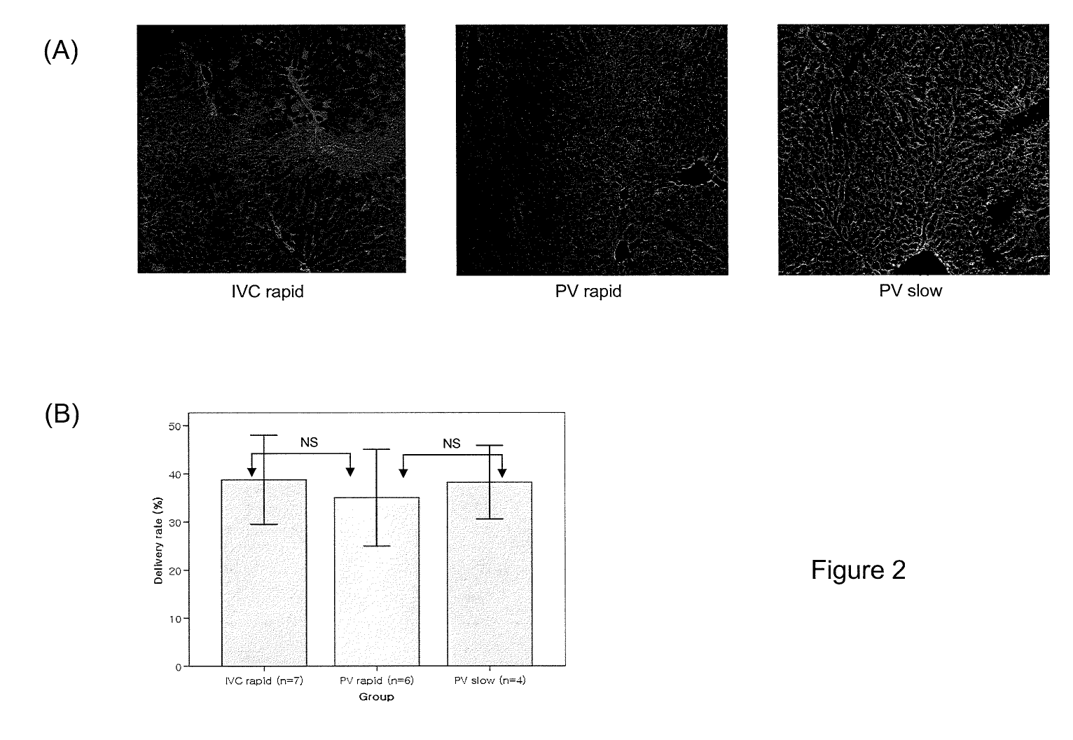

Tissue sections were collected at 1 hour after hydrodynamic injection and counterstained with Alexa488-phalloidin (green). The merged images of the two channels are shown. Two rapid injection groups showed strong and intense uptake in cytoplasm and nucleus (bright dot in the center of cell). However, PV slow injection group showed relatively diffuse and weak staining (FIG. 2A). Many periportal vein non-parenchymal cells (arrows) and perihepatic vein or sinusoidal endothelial cells (arrow heads) were positive in all groups (FIG. 2A).

The morphological delivery area between two groups was similar: 38.8±9.2% in the IVC group and 35.0±10.0% in the PV injection group (FIG. 2B). Trypan blue uptake (cells per HPF) in the IVC injection group was 43.4±25.5/HPF and in the PV group was 23.5±14.7/HPF, however the difference was not statistically significant (FIG. 2B).

### Example 3

### Delivery Rate and Toxicity According to Injection Speed

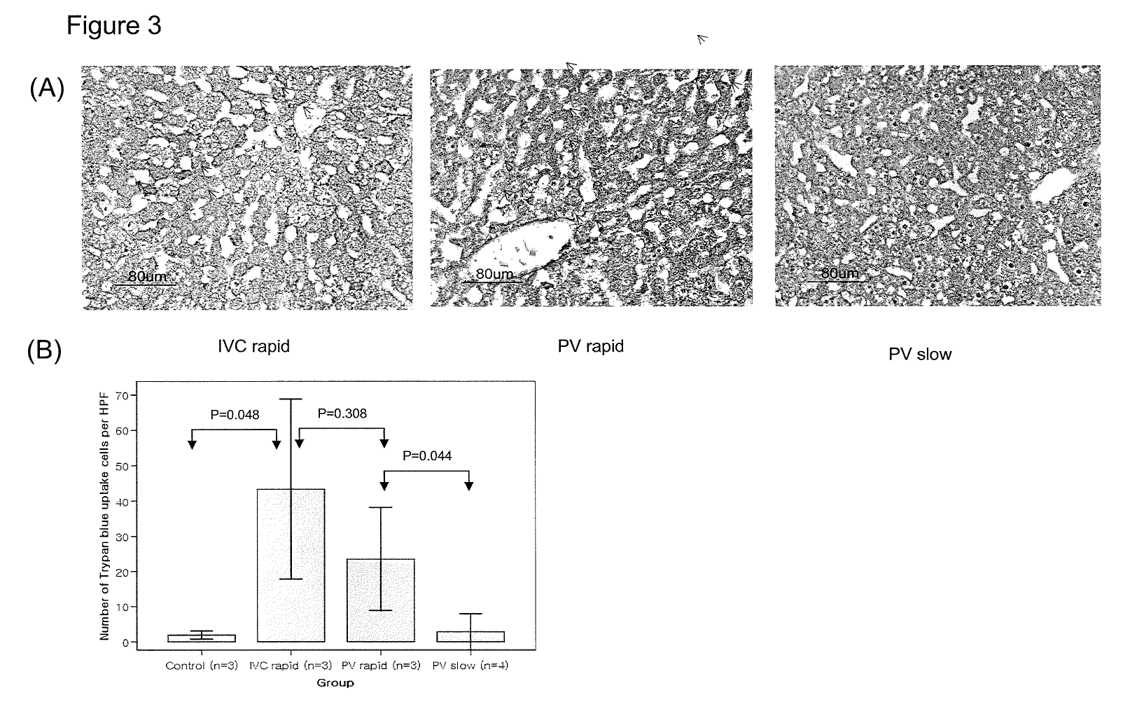

Livers were harvested and within 10 minutes of cold preservation, siRNA was injected in HTK via the portal vein using various methods as described above. The inferior vena cava and portal vein route were tested further in terms of injection speed (“IVC-rapid” vs “PV-slow” vs “PV rapid”). The PV-slow group showed a similar delivery rate to the PV-rapid group (38.2±7.7% vs. 35.0±10.0% respectively, p=0.609) (FIG. 3B). However, the pattern of delivery appeared slightly different. In the rapid injection group, most positive cells showed strong staining in the cytoplasm and nucleus (FIG. 2A).

In the slow injection group, some cells showed strong staining in the cytoplasm and nucleus, however, most cells showed only relatively weak staining (FIG. 2A). In both groups, most sinusoidal lining cells and periportal non-parenchymal cells were strongly positive.

Cold Trypan blue solution (40-50 ml of 200 mmol) was perfused via the portal vein of the liver graft at 1 hour after hydrodynamic injection, followed by 30-40 ml of cold 4% paraformaldehyde perfusion 5 mm frozen sections were cut and counterstained with Eosin. Three representative fields were selected from each lobe and Trypan blue positive cells were counted under the microscope (×200). The mean number of positive cells from three different lobes was considered as overall positive number per high power field (HPF). Many Trypan blue uptake cells were observed in rapid injection groups (A). Most Trypan blue positive cells were sinusoidal endothelial cells (arrows). There was no difference between control (no injection) and the slow injection group. However, the rapid injection groups showed more Trypan blue positive cells than control and PV slow injection group. Eosin staining showed multiple Trypan blue positive cells in the sinusoidal endothelial lining, and few positive hepatocytes in the rapid injection group. Parenchymal cells with diluted cytoplasm were observed around vessels in the rapid injection group (FIG. 3A). The number of Trypan blue uptake cells in the PV-slow injection group was 2.8±5.2/HPF, which was similar to control (1.9±1.2/HPF, p=0.780) and lower than the PV-rapid injection group (23.5±14.7/HPF, p=0.044) (FIG. 3B). These data demonstrate that slow injection results in less tissue damage and differential uptake pattern than the rapid injection methods.

### Example 4

### Survival and Degree of Injury after Transplantation

Livers were perfused with ice cold HKT and harvested as described above. Ice cold HTK containing siRNA was administered within 10 minutes of harvest using one of the three methods described above (“IVC-rapid” vs “PV-slow” vs “PV rapid”). Livers were maintained on ice for 45 minutes prior to transplantation into recipient rats.

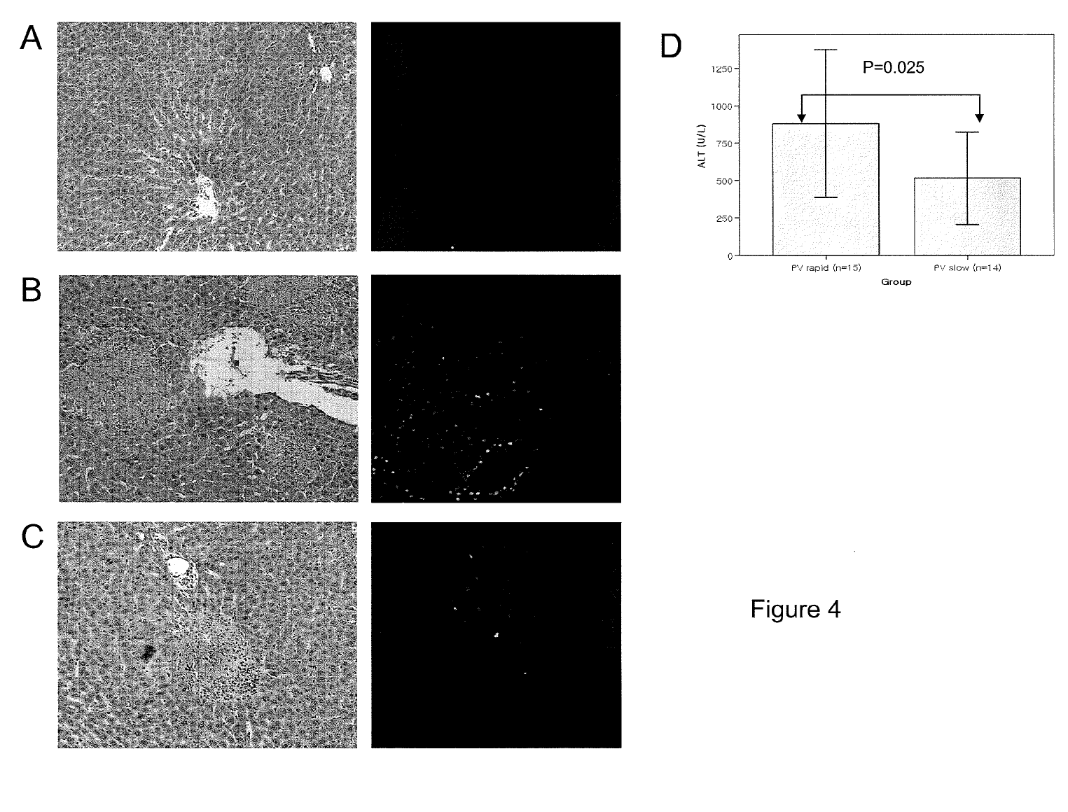

All transplanted animals in both groups survived the 24 hours after transplantation prior to sacrifice. H&E and TUNEL staining showed rare focal necroses around the portal vein in the rapid injection group at 24 hours after transplantation (FIG. 4A). This is correct, it is depicted by FIG. 4a The necrotic area consisted of about 5-10% of the liver. In the slow injection group, only a few small focal necroses were observed (FIG. 4C) by either H&E or TUNEL. In the rapid injection group, several TUNEL positive cells were observed in the periportal area and occasional endothelial cells were also positive for TUNEL staining. However, few TUNEL positive cells were observed in the slow group. Mean ALT levels 24 hours after transplantation were less than 1,000 U/L in both groups, however, the slow injection group showed significantly lower ALT level than the rapid group (516±309 vs. 881±492, p=0.025) (FIG. 4D). These data demonstrate decreased cell death and improved liver function after slow injection as compared to rapid injection.

### Example 5

### Functional Delivery Rate at 24 Hours after Transplantation According to Injection Speed

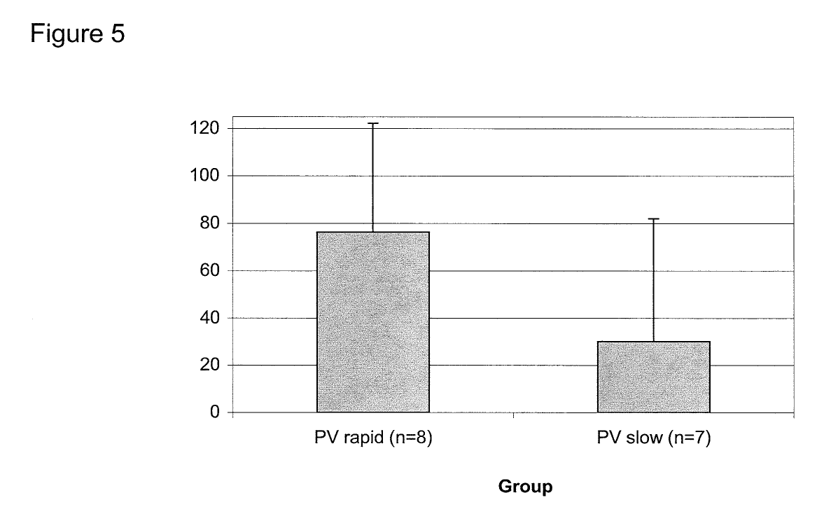

Functional outcomes at 24 hours after transplantation according to injection methods were assayed. Hepatocytes were isolated from transplanted livers using a two-step collagenase perfusion and differential centrifugation technique. Cyclophylin B mRNA relative expression levels were assayed by RT-PCR using RNA from hepatocytes. LacZ siRNA treated animals using the same delivery method (6 animals each) were used as control for each group. The PV-slow group showed better functional knockdown than PV-rapid group: 69.8±51.7% vs. 23.7±46.0% (FIG. 5). These data demonstrate that siRNA knockdown is more effective using the slow injection method as compared to the rapid injection method.

### Example 6

### Knockdown of HCV Expression Using siRNA

We have established a cell culture system in which to measure HCV replication and with which to develop novel small interfering RNAs effective in decreasing HCV replication (Lohmann et al., 1999. Science. 285:110-3). Human hepatoma cells (Huh7) stably transfected with a truncated HCV replicon (“Clone B”) were established and maintained in culture. A quantitative PCR-based assay was developed which was capable of detecting between approximately one hundred and one trillion copies of HCV in a linear range.

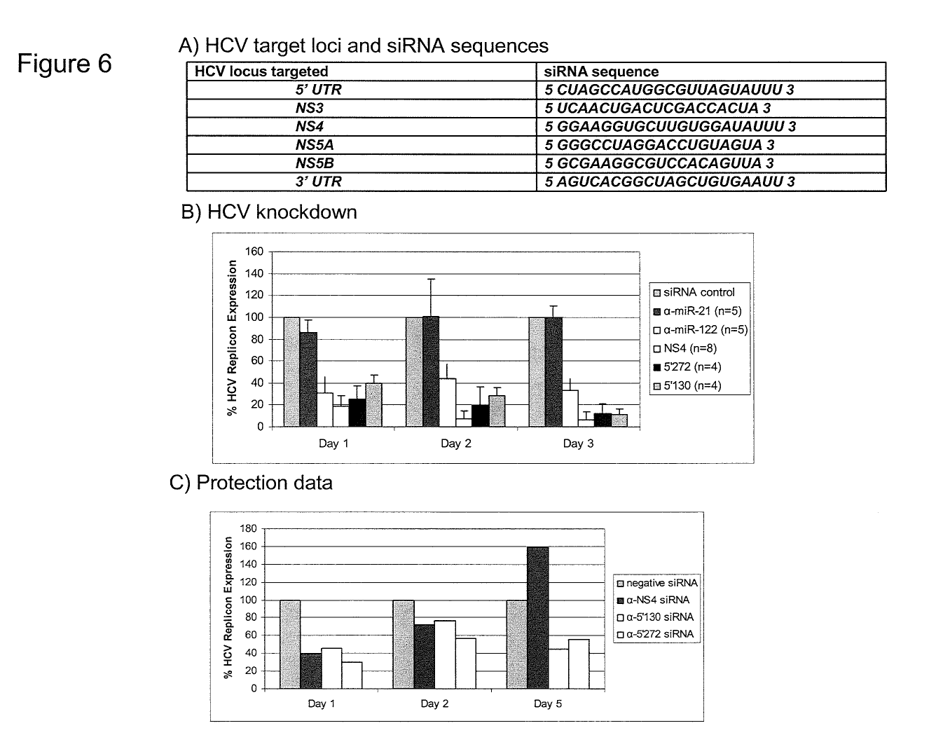

We designed several novel anti-HCV siRNAs based on areas of high homology between the known six genotypes of HCV (see FIG. 6A). We then treated HCV infected hepatocytes with 200 μg of our siRNAs and measured rates of HCV replication (FIGS. 6A and B). Shown here are the three day results of quantitative PCR based assays of cellular HCV levels in response to treatment with our novel anti-HCV siRNAs. In addition to the NS4 siRNA targeted to HCV, siRNA targeted to miR-122 (Dharmacon) and were also able to substantially decrease HCV replicon expression as compared to negative control siRNA. Several were potent in inhibiting HCV replication on the order of 95-99%. These data demonstrate that siRNA delivered to hepatocytes can inhibit HCV replication, treating HCV infection.

### Example 7

### SiRNAs Protect Hepatocytes from HCV Infection

The siRNAs in Example above were used to protect naïve hepatocytes from HCV infection. In a series of similarly designed experiments we transfected siRNAs into hepatocytes prior to exposure to HCV. Lower levels of HCV were subsequently detected in cells that had been treated with anti-HCV siRNAs targeted to HCV, as compared to control siRNAs (FIG. 6C). The results correlate well with the HCV replicon expression inhibition results. These data demonstrate that siRNA specific to HCV delivered to a naïve cell can prevent HCV infection.

### Example 8

### Knockdown of HCV Expression Using shRNA Delivered in a Viral Vector

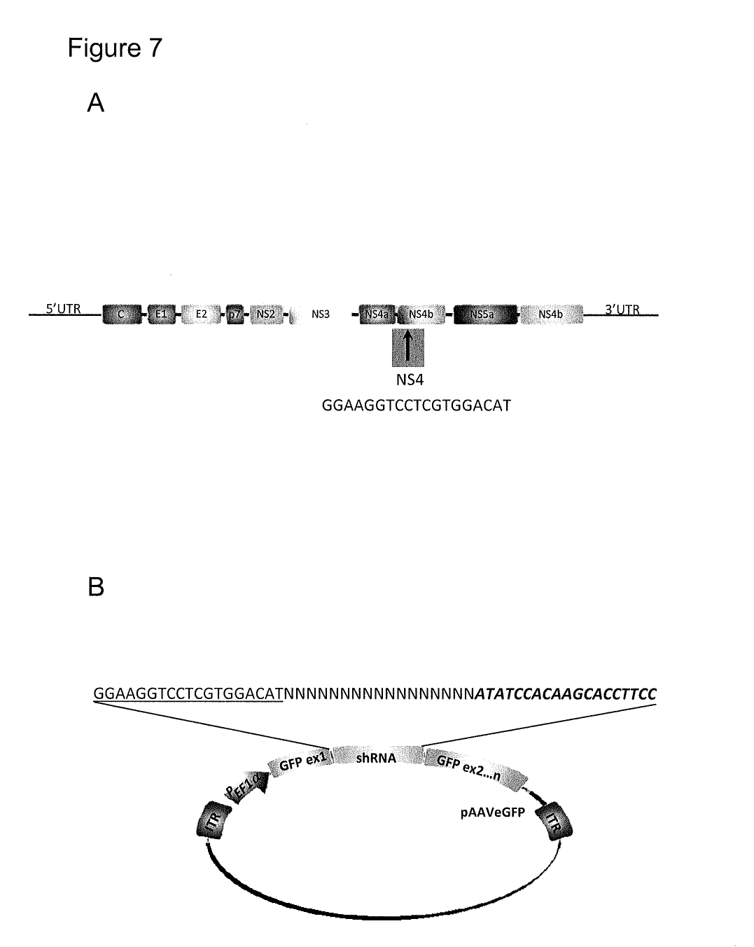

Using the HCV culture system above, an siRNA sequence used above was expressed from a recombinant AAV vector as shRNA fused to green fluorescent protein (GFP) (shown schematically in FIGS. 7A and B). AAV plasmid vectors carrying the indicated sequence were constructed and expressed using routine methods. Constructs were sequenced to confirm the identity of the expression construct.

Huh7 cells were infected with an AAV vector (Titer: 1×10¹² particles/ml, 200 microliters used per experiment) expressing an HBV sequence or the NS4-GFP sequence shown in FIG. 7B using routine methods. After the number of days indicated, cells were harvested and total RNA was isolated. Equal amounts of RNA were loaded into wells, separated by electrophoresis, transferred to nitrocellulose, and probed as indicated.

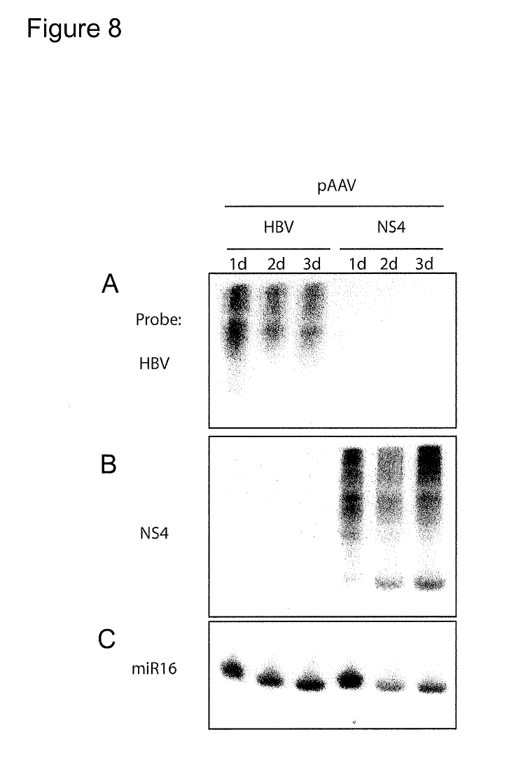

FIGS. 8A and B demonstrate expression of each of the constructs in the liver cells with sustained expression for three days. FIG. 8C is a loading control. Similar results were obtained when cells were transfected with 200 μg of plasmid DNA. Target knockdown was observed for two weeks providing evidence of durable expression.

### Example 9

### Determination of Transfection Efficiency Using the AAV NS4-GFP Plasmid Vector to Allow for the Assessment of Target Knockdown

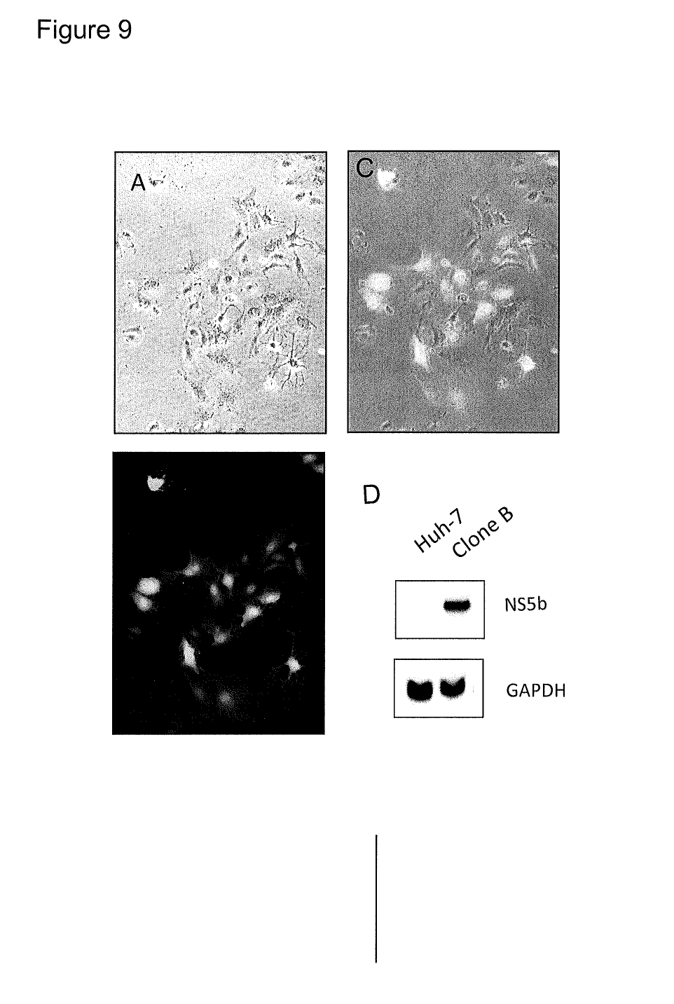

Huh7 cells were transfected with the AAV NS4-GFP plasmid vector (200 μg). After three days, cells were imaged to determine transduction levels. FIGS. 9A and B show brightfield and fluorescence images of the cells, respectively. The merged image is shown in FIG. 9C. Transduction efficiency was determined to be about 60% three days after infection.

Clone B replication was further determined by Northern blot (FIG. 9D). Briefly, Huh7 cells or Huh7 cells infected with HCV. After three days, total RNA was harvested, subject to electrophoresis, transferred to nitrocellulose, and probed as indicated. As shown in the top panel, the replicon specific NS5b sequence could only be detected in the cells infected with Clone B. The GAPDH blot is shown as a loading control (FIG. 9D). This demonstrates that HCV infection can be detected by Northern blot using the NS5b probe.

### Example 10

### Determination of Target Knockdown Using the AAV NS4-GFP Plasmid Vector in View of Transduction Efficiency

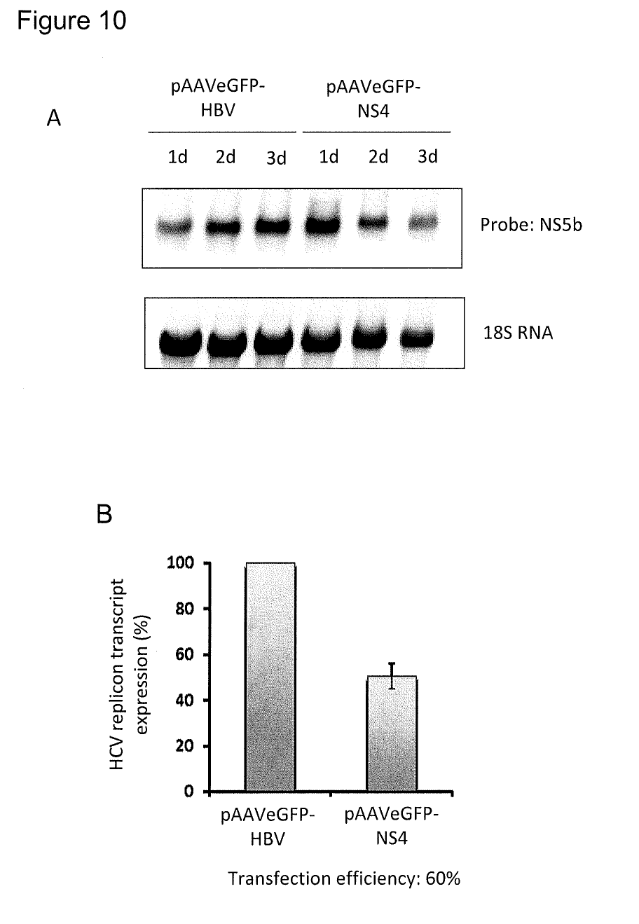

Having determined the transduction efficiency of Huh7 cells using the AAV NS4-GFP vector, knockdown of HCV infection was determined Clone B infected Huh7 cells were transfected with the AAVeGFP-HBV vector or the pAAVeGFP-NS4 vector (200 μg) as in the previous example. After three days, cells were harvested, total RNA was isolated, resolved by electrophoresis, and transferred to nitrocellulose. The blot was probed with the NS5b probe to detect HCV infection and with an 18S RNA probe as a loading control (FIGS. 10A and B). Quantitation of the blot and analysis by qPCR demonstrated about a 50% knockdown in viral expression. As the transfection efficiency was found to be only about 60%, a knockdown of expression of 50% represents a substantial decrease in viral replication.

### Example 11

### Expression of GFP in Mouse Liver Using an scAAV8 EF1a GFP Expression Vector in Wild Type Mouse Liver

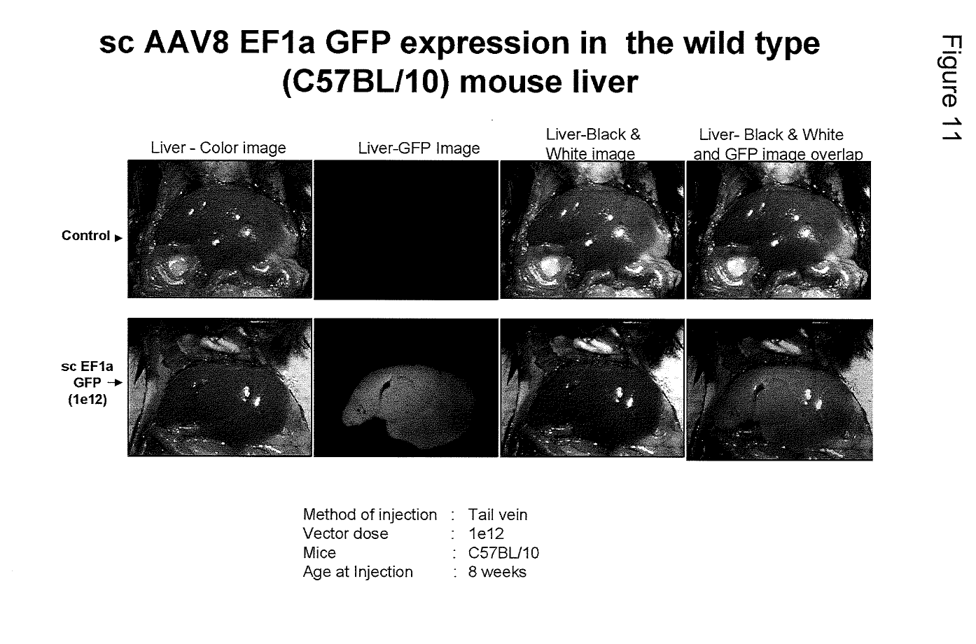

Having demonstrated target specific knockdown of HCV using both siRNA and shRNA, the AAV construct for the expression of e.GFP was administered systemically to mice (n=10 per group) by tail vein injection (10¹² viral particles). After 8 weeks, mice were sacrificed, and livers were exposed and imaged for GFP expression. FIG. 10 shows 8 weeks after administration of the AAV8-EF1a-eGFP viral vector, eGFP expression can be readily observed (FIG. 11). Further analysis of the liver demonstrates that nearly 100% of hepatocytes express eGFP. These data demonstrate that AAV vectors can be used to efficiently deliver coding sequences to the liver and that expression in the liver is sustained in vivo for an extended period (through at least 6 months).

### Example 12

### Hepatic Ex Vivo Gene Transfer

Routine organ procurement takes place with cold preservation of a liver destined for a patient with HCV cirrhosis. The vasculature/direction of perfusion of the liver (e.g., portal vein, hepatic artery, or inferior vena cava) is noted.

On the backtable, typically in the operating room of the recipient, under cold conditions, the liver is injected with 1-10 mls of nucleic acid in an appropriate carrier, e.g., high titer AAV (1×10¹²⁻¹⁴) or non-viral DNA expression construct, directing expression of shRNAs of interest, i.e. anti-HCV. Injection is via portal vein and/or hepatic artery, preferably perfusing the liver in the same direction in which it was perfused during cold preservation. Alternatively naked siRNAs (approx 1-10 mg/kg) of corresponding sequence suspended in preservation solution is injected, preferably via the portal vein. This is accomplished utilizing a sterile 10 cc syringe and an 18 gauge angiocatheter. The slow injection technique is utilized in an attempt to keep hydrodynamic injection pressures around 10 mm Hg. Injection time is typically around 3-5 minutes. Alternatively, both naked siRNAs and virus are injected as a combined cocktail.

Cold preservation continues until the patient has undergone hepatectomy and is ready for transplant. Liver Transplant ensues in standard fashion.

All patents, patent applications, GenBank numbers in the version available as of the priority date of the instant application, and published references cited herein are hereby incorporated by reference in their entirety as if they were incorporated individually. While this invention has been particularly shown and described with references to preferred embodiments thereof, it will be understood by those skilled in the art that various changes in form and details may be made therein without departing from the scope of the invention encompassed by the appended claims.
1. McCaffrey et al., RNA interference in adult mice. Nature 2002; 418:38-39.
2. Lewis et al., Efficient delivery of siRNA for inhibition of gene expression in postnatal mice. Nat Genet 2002; 32:107-108.
3. Lewis D L, Wolff J A. Systemic siRNA delivery via hydrodynamic intravascular injection. Adv Drug Deliv Rev 2007.
4. Inoue et al., Gene therapy for organ grafts using rapid injection of naked DNA: application to the rat liver. Transplantation 2004; 77:997-1003.
5. Sato et al. Gene silencing in rat-liver and limb grafts by rapid injection of small interference RNA. Transplantation 2005; 79:240-243.
6. Tsoulfas et al., et al., Hydrodynamic plasmid DNA gene therapy model in liver transplantation. J Surg Res 2006; 135:242-249.
7. Sato et al., Small interfering RNA delivery to the liver by intravenous administration of galactosylated cationic liposomes in mice. Biomaterials 2007; 28:1434-1442.
8. Herweijer H, Wolff J A. Gene therapy progress and prospects: hydrodynamic gene delivery. Gene Ther 2007; 14:99-107.
9. Lewis D L, Wolff J A. Delivery of siRNA and siRNA expression constructs to adult mammals by hydrodynamic intravascular injection. Methods Enzymol 2005; 392:336-350.
10. Kamada N, Calne R Y. A surgical experience with five hundred thirty liver transplants in the rat. Surgery 1983; 93:64-69.
11. Smedsrod B, Pertoft H. Preparation of pure hepatocytes and reticuloendothelial cells in high yield from a single rat liver by means of Percoll centrifugation and selective adherence. J Leukoc Biol 1985; 38:213-230.
12. Maemura et al. Reactive oxygen species are essential mediators in antigen presentation by Kupffer cells. Immunol Cell Biol 2005; 83:336-343.
13. Pfaffl et al., Relative expression software tool (REST) for group-wise comparison and statistical analysis of relative expression results in real-time PCR. Nucleic Acids Res 2002; 30:e36.
14. Zhang et al., High levels of foreign gene expression in hepatocytes after tail vein injections of naked plasmid DNA. Hum Gene Ther 1999; 10:1735-1737.
15. Eastman et al. Development of catheter-based procedures for transducing the isolated rabbit liver with plasmid DNA. Hum Gene Ther 2002; 13:2065-2077.
16. Budker et al., Mechanism of plasmid delivery by hydrodynamic tail vein injection. II. Morphological studies. J Gene Med 2006; 8:874-888.
17. Sebestyen et al. Mechanism of plasmid delivery by hydrodynamic tail vein injection. I. Hepatocyte uptake of various molecules. J Gene Med 2006; 8:852-873.
18. Rossmanith et al., Cellular gene dose and kinetics of gene expression in mouse livers transfected by high-volume tail-vein injection of naked DNA. DNA Cell Biol 2002; 21:847-853.
19. Budker et al., Hypothesis: naked plasmid DNA is taken up by cells in vivo by a receptor-mediated process. J Gene Med 2000; 2:76-88.
20. Song et al. RNA interference targeting Fas protects mice from fulminant hepatitis. Nat Med 2003; 9:347-351.
21. Li et al. Alleviation of ischemia-reperfusion injury in rat liver transplantation by induction of small interference RNA targeting Fas Langenbecks Arch Surg 2007; 392:345-351.
22. Liu F, Huang L Improving plasmid DNA-mediated liver gene transfer by prolonging its retention in the hepatic vasculature. J Gene Med 2001; 3:569-576.
23. Kawabata et al., The fate of plasmid DNA after intravenous injection in mice: involvement of scavenger receptors in its hepatic uptake. Pharm Res 1995; 12:825-830.
24. Yoshino et al., Naked plasmid DNA transfer to the porcine liver using rapid injection with large volume. Gene Ther 2006; 13:1696-1702.
25. Prosser et al., Molecular therapy for hepatic injury and fibrosis: where are we? World J Gastroenterol 2006; 12:509-515.
26. Ke et al., Gene therapy in liver ischemia and reperfusion injury. Curr Pharm Des 2006; 12:2969-2975.
27. Brandwijk et al., Targeted gene-delivery strategies for angiostatic cancer treatment. Trends Mol Med 2007; 13:200-209.
28. Budker et al., Naked DNA delivered intraportally expresses efficiently in hepatocytes. Gene Ther 1996; 3:593-598.
29. Zhang et al., Regional hydrodynamic gene delivery to the rat liver with physiological volumes of DNA solution. J Gene Med 2004; 6:693-703.
30. Zhang et al., Expression of naked plasmid DNA injected into the afferent and efferent vessels of rodent and dog livers. Hum Gene Ther 1997; 8:1763-1772.

1. A method of ex vivo hepatic nucleic acid delivery comprising:
obtaining a liver; and
injecting the nucleic acid in a carrier into vasculature of the liver at a low pressure 2-12 mmHg, whereby the nucleic acid is delivered to hepatic tissue.

2. The method of claim 1, wherein the volume of the nucleic acid in the carrier is about 0.33 ml to about 20 ml per kg of liver to receive the nucleic acid in carrier.

3. The method of claim 1, wherein the nucleic acid in the carrier is injected at a rate of 5-15 ml/minute.

4. The method of claim 1, wherein the method further comprises perfusing the liver with cold pharmaceutically acceptable prior to the injection of the nucleic acid.

5. The method of claim 1, wherein the nucleic acid in the carrier is injected into the portal vein, hepatic artery, or inferior vena cava.

6. The method of claim 1, wherein the nucleic acid in the carrier is retained in the liver for at least 15 minutes.

7. The method of claim 1, wherein the nucleic acid comprises an siRNA.

8. The method of claim 1, wherein the nucleic acid comprises an antisense nucleic acid.

9. The method of claim 1, wherein the nucleic acid comprises an shRNA.

10. The method of claim 1, wherein the nucleic acid is targeted to a hepatitis virus.

11. The method of claim 10, wherein the hepatitis virus comprises a hepatitis C virus.

12. The method of claim 11, wherein at least a portion of the nucleic acid specifically hybridizes to at least 15 contiguous nucleic acids of a sequence selected from the group consisting of:

| | |
|---|---|
| 5′-CUAGCCAUGGCGUUAGUAUUU-3′, | (SEQ ID NO: 1) |
| 5′-UCAACUGACUCGACCACUA-3, | (SEQ ID NO: 2) |
| 5′-GGAAGGUGCUUGUGGAUAUUU-3′, | (SEQ ID NO: 3) |
| 5′-GGGCCUAGGACCUGUAGUA-3′, | (SEQ ID NO: 4) |
| 5′-GCGAAGGCGUCCACAGUUA-3′, | (SEQ ID NO: 5) |
| and | |
| 5′-AGUCACGGCUAGCUGUGAAUU-3′. | (SEQ ID NO: 6) |

13. The method of claim 9, wherein the antisense nucleic acid or the shRNA is encoded by an expression construct.

14. The method of claim 13, wherein the expression construct is present in a vector selected from the group consisting of a plasmid vector, an adenoviral (Ad) vector, an adeno-associated viral vector (AAV), a lentiviral vector, and a herpes simplex viral (HSV) vector.

15. The method of claim 14, wherein the AAV viral vector is selected from the group consisting of an AAV2 viral vector, an AAV8 vector, an AAV9 vector, a hybrid AAV2/4 viral vector, and a hybrid AAV2/5 viral vectors.

16. The method of claim 14, wherein nucleic acid in the AAV viral vector is self-complementary.

17. The method of claim 14, wherein the viral vector is replication competent.

18. The method of claim 14, wherein the viral vector is replication incompetent.

19. The method of claim 13, wherein the expression construct comprises a promoter sequence selected from the group consisting of a cytomegalovirus (CMV) promoter, a β-globin promoter, chicken β-actin (CBA) promoter, and small chicken β-actin (smCBA) promoter, and an EF-1α promoter.

20. The method of claim 14, wherein the number of viral particles delivered comprises 10¹⁰-10¹⁴ particles per 1.5 kg liver.

21-23. (canceled)

24. The method of claim 1, further comprising transplanting the liver into a subject.

25. The method of claim 24 further comprising monitoring the subject for HCV infection.

26-29. (canceled)
